(19) United States
(12) Patent Application Publication
(10) Pub. No.: US 2015/0196687 A1
(43) Pub. Date: Jul. 16, 2015

(54) Compositions and Uses to Govern Cancer Cell Growth

(71) Applicant: MASSACHUSETTS INSTITUTE OF TECHNOLOGY, CAMBRIDGE, MA (US)

(72) Inventors: Joseph W. Franses, Brookline, MA (US); Elazer R. Edelman, Brookline, MA (US); Angelo Manuel De Almeida Cardoso, Indianapolis, IN (US); Helen Marie Nugent, Needham, MA (US)

(21) Appl. No.: 14/592,395

(22) Filed: Jan. 8, 2015

Related U.S. Application Data
(63) Continuation of application No. 14/341,220, filed on Jul. 25, 2014, now abandoned.
Application No. 14/341,220 is a continuation of application No. 13/147,695, filed on Oct. 18, 2011, now abandoned.
(60) Provisional application No. 61/149,885, filed on Feb. 4, 2009.
(60) Provisional application No. 61/252,358, filed on Oct. 16, 2009.

(51) Int. Cl. A61L 27/38; A61L 27/50; A61L 27/40

(52) U.S. Cl. CPC A61L 27/3808; A61L 27/40; A61L 27/50; A61L 2400/18

(57) ABSTRACT

The invention relates to composition and a method of using the composition for modulting proliferation, invasiveness, the expression of a biomarker of an abnormal cell, of reducing the risk of patient cell becoming abnormal, or of modulating proliferation of a carcinoma-associated fibroblast or of a tumor-associated macrophage. The invention also relates to a method of culturing the composition to produce molecules that modulate abnormal cell proliferation, invasiveness, or metastasis. The composition comprises a biocompatible matrix and cells engrafted thereon.

### FIELD

The invention relates to the field of cancer biology. In particular, it relates to methods and compositions for modulating and managing cancer cell virulence and growth.

### BACKGROUND

Cancer remains a leading cause of morbidity and mortality with approximately 1.4 million new cases and 560,000 deaths in the United States alone in 2007. (Peto, Nature, 411:390-395 (2001); Jernal, CA Cancer J. Clin., 57:43-66 (2007). Emerging insights into cancer's pathobiology and the potential of novel therapies have only modestly reduced these numbers. Moreover, cancer therapy is itself potentially devastating. Surgical tumor resection, systemic chemotherapy, and regional radiation therapy kill cancer cells, (Schmitt, J. Pathol., 187:127-137 (1999)), but they also cause substantial damage to the body and have serious side effects. Furthermore, many treatments ultimately fail at their principal goal of prolonging life. Given the problems of current cancer therapies, there is still a need for better therapies.

### SUMMARY OF THE INVENTION

This Summary is provided merely to introduce certain concepts and not to identify any key or essential features of the claimed subject matter.

In one aspect, the invention relates to a method of modulating proliferation of an abnormal cell. The method comprises providing an implantable material in the vicinity of an abnormal cell, wherein the implantable material comprises a biocompatible matrix and cells engrafted thereon and wherein the implantable material is in an amount effective to modulate proliferation of the abnormal cell.

In another aspect, the invention relates to a method of modulating invasiveness of an abnormal cell. The method comprises providing an implantable material in the vicinity of an abnormal cell, wherein the implantable material comprises a biocompatible matrix and cells engrafted thereon and wherein the implantable material is in an amount effective to modulate invasiveness of the abnormal cell. According to one embodiment, invasiveness is migration or metastasis.

In a further aspect, the invention relates to a method of altering expression of a biomarkers of an abnormal cell. The method comprises the step of providing an implantable material in the vicinity of an abnormal cell, wherein the implantable material comprises a biocompatible matrix and cells engrafted thereon and wherein the implantable material is in an amount effective to alter expression of the biomarker of the abnormal cell.

According to one embodiment, the biomarker is selected from the group consisting of: p53, pRb, HIIF-1α, NF-κB, SNAIL, ABCG2, CD133, MMP2, MMP9, HERR2, CD44, STAT1, STAT2, STAT3, STAT4, STAT5, STAT6, JAK1, JAK2, Twist, Snail, Slug, Sip1, Ki67, PCNA, N-cadherin, fibronectin, VEGF, FGF, HGF, EGF, IGF, TGF-beta, BMP, versican, perlecan, one or more genes listed in FIG. 20, other cancer stem cell markers, other virulence markers, other metastasis markers, and combinations of any of the foregoing biomarkers.

According to various embodiments, the abnormal cell is selected from the group consisting of: tumor cell, cancer cell, precancer cell, neoplastic cell, hyperplastic cell, cancer stem cell, progenitor cell, metastasizing or metastatic cell, a combination of any of the foregoing abnormal cells, an abnormal tissue, and cells within an abnormal tissue. According to additional embodiments, the implantable material is provided near, adjacent or in contact with the abnormal cell, the implantable material is provided at a site remote from the abnormal cell, and/or the implantable material exerts a paracrine, endocrine, or other biochemical effect on the abnormal cell. According to an additional embodiment, the cells are endothelial cells, endothelial-like cells, epithelial cells, epithelial-like cells, endothelial progenitor cells, stem cells, analogs of any of the foregoing, or a co-culture of at least two of the foregoing.

In another aspect, the invention relates to a method of modulating proliferation or recruitment of a carcinoma-associated fibroblast or a tumor-associated macrophage. The method comprises providing an implantable material in the vicinity of a carcinoma having a carcinoma-associated fibroblast, wherein the implantable material comprises a biocompatible matrix and cells engrafted thereon and wherein the implantable material is in an amount effective to modulate proliferation of the carcinoma-associated fibroblast or the tumor-associated macrophage.

According to various embodiments, the cells are endothelial cells, endothelial-like cells, epithelia cells, epithelial-like cells, endothelial progenitor cells, stem cells, analogs of any of the foregoing, or a co-culture of at least two of the foregoing.

In a further aspect, the invention relates to a method of producing molecules that modulate abnormal cell proliferation, invasiveness, migration, or metastasis. The method comprises culturing cells engrafted on a biocompatible matrix, wherein the cells produce molecules that modulate abnormal cell proliferation, invasiveness, migration, or metastasis.

According to various embodiments, the cells are endothelial cells, endothelial-like cells, epithelial cells, epithelial-like cells, endothelial progenitor cells, stem cells, analogs of any of the foregoing, or a co-culture of at least two of the foregoing. The invention further relates to the cultured cells or a cell culture effluent produced according to the method or purified molecules as produced by the cells or associated with the effluent.

In a further aspect, the invention relates to a method of treating neoplasia, neoplastic or dysplastic growth. The method comprises providing an implantable material in the vicinity of a neoplasm site, wherein the implantable material comprises a biocompatible matrix and cells engrafted thereon and wherein the implantable material is in an amount effective to treat the neoplasm site.

In an additional aspect, the invention relates to a method of reducing the risk of reducing the risk of a patient cell becoming abnormal. The method comprises providing an implantable material in the vicinity of a patient cell, wherein the implantable material comprises a biocompatible matrix and cells engrafted thereon and wherein the implantable material is in an amount effective to reduce the risk of the patient cell becoming abnormal.

According to various embodiments, the effective amount modulates neoplastic cell differentiation, proliferation or migration at, near or adjacent the neoplasm site, the effective amount modulates neoplasm smooth muscle cell differentiation, proliferation or migration at, near or adjacent the neoplasm site, the effective amount modulates neoplasm vascularization at, near or adjacent the neoplasm site, and/or the effective amount modulates neoplastic invasion at, near or adjacent the neoplasm site.

According to various embodiments, providing the implantable material is accomplished by percutaneously depositing the implantable material at, near, adjacent or contacting the neoplasm site. According to additional embodiments, the cells are endothelial cells, endothelial-like cells, epithelial cells, epithelial-like cells, endothelial progenitor cells, stem cells, analogs of any of the foregoing, or a co-culture of at least two of the foregoing.

In a further aspect, the invention relates to a method of treating neoplasia. The method comprises contacting a neoplastic cell with an anti-neoplastic factor, wherein the factor is present in an effluent derived from a biocompatible matrix and cells engrafted thereon or therein and wherein the factor is provided in an amount effective to modulate, modulate or retard the growth of the neoplastic cell.

According to one embodiment, the neoplastic cell is contacted with an effective amount of the effluent. According to an additional embodiment, the neoplasm is a benign neoplasm or a malignant neoplasm.

In a further aspect, the invention relates to a method for reducing the risk of neoplasia or dysplasia. The method comprises providing an implantable material to a subject at risk for developing neoplasia, wherein the implantable material comprises a biocompatible matrix and cells engrafted thereon which reduces the risk of the subject developing neoplasia. According to one embodiment, the implantable material is provided in the vicinity of a cell at risk for becoming neoplastic or dysplastic. According to a further embodiment, the cell at risk for becoming neoplastic comprises the BRCAI allele.

According to various embodiment, the implantable material exerts a paracrine effect on the neoplasia. According to additional embodiments, the neoplasia is selected from the group consisting of: carcinoma (including adenocarcinoma, aquamous cell carcinoma or other subtypes of carcinoma derived from epithelial tissues including but not limited to, lung, breast, pancreas, colon, stomach, esophagus, bladder, prostate, endometrium, ovary, cervix, larynx, oropharynx, skin), sarcoma (including but not limited to leiomyosarcoma (derived from smooth muscle) rhabdomyosarcoma (striated muscle), chondrosarcoma (cartilage), angiosarcoma (endothelial cells), fibrosarcoma (fibroblasts), liposarcoma (adipocytes), osteosarcoma (bone), synovial sarcoma (synovium)), hematopoietic malignancies (including but not limited to leukemia (derived from any blood-forming element), lymphoma (any blood-forming element), or myeloma (plasma cells)), neuroectodermal tumors (including but not limited to gliomas, glioblastomas, neuroblastomas, schwannomas, and medulloblastomas), neural crest-derived cancers (including but not limited to small-cell lung carcinomas, melanomas, pheochromocytomas), and anaplastic (dedifferentiated) cancers. According to a further embodiment, the effective amount reduced neoplastic metastasis or paraneoplasia.

In another aspect, the invention relates to a composition suitable for modulating proliferation or invasiveness of an abnormal cell, the composition comprising a biocompatible matrix and anchored or embedded endothelial cells, endothelial-like cells, epithelial cells, epithelial-like cells, endothelial progenitor cells, stem cells, analogues thereof, or a co-culture of at least two of the foregoing, wherein said composition is in an amount effective to modulate the proliferation or invasiveness of the abnormal cell.

In a further aspect, the invention relates to a composition suitable for modulating proliferation of a carcinoma-associated fibroblast or a tumor-associated macrophage or other tumor or cancer-associated stromal cellular element, the composition comprising a biocompatible matrix and anchored or embedded endothelial cells, endothelial-like cells, epithelial cells, epithelial-like cells, endothelial progenitor cells, stem cells, analogues thereof, or a co-culture of at least two of the foregoing, wherein said composition is in an amount effective to modulate the proliferation of a carcinoma-associated fibroblast or a tumor-associated macrophage.

In a further aspect, the invention relates to a composition suitable for treating neoplasia, the composition comprising a biocompatible matrix and anchored or embedded endothelial cells, endothelial-like cells, epithelial cells, epithelial-like cells, endothelial progenitor cells, stem cells, analogues thereof, or a co-culture of at least two of the foregoing, wherein said composition is in an amount effective to treat the neoplasia.

In another aspect, the invention relates to a composition suitable for reducing the risk of a patient cell becoming abnormal, the composition comprising a biocompatible matrix and anchored or embedded endothelial cells, endothelial-like cells, epithelial cells, epithelial-like cells, endothelial progenitor cells, stem cells, analogues thereof, or a co-culture of at least two of the foregoing, wherein said composition is in an amount effective to reduce the risk of the patient cell becoming abnormal.

According to various embodiment, the biocompatible matrix is a flexible planar material or a flowable composition. Further, the cells may comprise a population of cells selected from the group consisting of near-confluent cells, confluent cells and post-confluent cells. According to a further embodiment, the cells are not exponentially growing cells, the cells are engrafted to the biocompatible matrix via cell to matrix interactions, and/or the composition further comprises a second therapeutic agent.

### BRIEF DESCRIPTION OF DRAWINGS

The present teachings described herein will be more fully understood from the following description of various illustrative embodiments, when read together with the accompanying drawings. It should be understood that the drawings described below are for illustration purposes only and are not intended to limit the scope of the present teachings in any way.

FIGS. 1A and 1B show cell growth curves, in accordance with an illustrative embodiment.

FIGS. 2A and 2B show proliferation curves for MDA-MB-231 cells (FIG. 2A) and A549 cells (FIG. 2B) grown in endothelial cell-conditioned media, in accordance with an illustrative embodiment.

FIGS. 3A-3C show graphs depicting cancer cell proliferation (FIG. 3A), gels depicting PCNA expression (FIG. 3B), and fluorescent images of K167 expression (FIG. 3C) in cancer cells grown in endothelial cell-conditioned media, in accordance with an illustrative embodiment.

FIGS. 4A-4E show graphs depicting cancer cell proliferation (FIG. 4A), graphs depicting cell cycle progression (FIG. 4B), a gel and a graph depicting expression and of cell cycle proteins (FIG. 4C), graphs depicting expression of signaling proteins (FIG. 4D) in cancer cells grown in endothelial cell-conditioned media, and a graph depicting cancer cell proliferation of cancer cells co-cultured with engrafted endothelial cells (FIG. 4E), in accordance with an illustrative embodiment.

FIG. 5 shows a graph depicting proliferation of MCF7 cells grown in media conditioned with engrafted endothelial cells, in accordance with an illustrative embodiment.

FIG. 6 shows a graph depicting proliferation of SK-LMS-lleiomyosarcoma cells grown in endothelial cell-conditioned media, in accordance with an illustrative embodiment.

FIG. 7 shows a graph depicting proliferation of NCI-520 cells grown in endothelial cell-conditioned media, in accordance with an illustrative embodiment.

FIG. 8 is a schematic depicting an invasion/migration assay, in accordance with an illustrative embodiment.

FIG. 9A-9E show a graph depicting cancer cell invasiveness (FIG. 9A), a graph depicting expression of pro-invasive genes and anti-invasive gene (FIG. 9B), a graph depicting cancer cell proliferation (FIG. 9C), a graph depicting cancer cell invasiveness (FIG. 9D) in cancer cells grown in endothelial cell-conditioned media, and a graph depicting cancer cell invasiveness of cancer cells grown in media conditioned with engrafted endothelial cells, in accordance with an illustrative embodiment.

FIGS. 10A-10C show gels depicting phosphorylation or expression of pro-tumorigenic signaling proteins (FIG. 10A), fluorescent images of NF-κB expression (FIG. 10B), and gels depicting phosphorylation or expression of pro-tumorigenic signaling proteins (FIG. 10C) in cancer cells grown in endothelial cell-conditioned media, in accordance with an illustrative embodiment.

FIGS. 11A-11C show a graph depicting TGF-β expression in endothelial cells (FIG. 11A), cancer cell proliferation of cancer cells grown in endothelial cell-conditioned media (FIG. 11B), and a chart listing exemplary genes differently expressed in cancer cells (FIG. 11C), in accordance with an illustrative embodiment.

FIG. 12 shows a lentivirus plasmid construct, in accordance with an illustrative embodiment.

FIGS. 13A-13C show graphs depicting reduction in perlecan expression (FIG. 13A), proliferation of endothelial cells (FIG. 13B), and endothelial cell tube formation (FIG. 13C) in perlecan shRNA knockdown endothelial cells, in accordance with an illustrative embodiment.

FIGS. 14A-14C show graphs depicting cancer cell proliferation (FIG. 14A), graphs depicting cancer cell invasiveness (FIG. 14B), and gels depicting phosphorylation of pro-tumorigenic signaling molecules (FIG. 14C) in cancer cells grown in media conditioned by perlecan knockdown endothelial cells, in accordance with an illustrative embodiment.

FIGS. 15A-15C show graphs depicting expression of cytokines (FIG. 15A), cancer cell proliferation (FIG. 15B), and cancer cell invasiveness (FIG. 15C) of cancer cells grown in media conditioned by perlecan knockdown endothelial cells, in accordance with an illustrative embodiment.

FIGS. 16A-16E show graphs depicting cancer cell proliferation (FIG. 16A), cancer cell invasiveness (FIG. 16B), and protein expression (FIGS. 16C-E) in cancer cells grown in media conditioned by perlecan knockdown endothelial cells, in accordance with an illustrative embodiment.

FIG. 17 shows a graph depicting cancer cell proliferation of cancer cells grown in media conditioned by perlecan knockdown endothelial cells, in accordance with an illustrative embodiment.

FIG. 18 shows a graph depicting cancer cell proliferation of cancer cells grown in media conditioned with engrafted endothelial cells, in accordance with an illustrative embodiment.

FIGS. 19A-19D show a schematic depicting an experimental design (FIG. 19A), a graph depicting in vivo reduction of tumor volume in response to implanted endothelial cells (FIG. 19B), a graph depicting the number of K167 expressing nuclei (FIG. 19C), and fraction cystic area of tumors (FIG. 19D), in accordance with an illustrative embodiment.

FIG. 20 is a table listing exemplary cancer marker genes, in accordance with an illustrative embodiment.

### DETAILED DESCRIPTION

As disclosed herein, the invention relates to the discovery that a cell-based therapy can be used to treat, heal, ameliorate, manage, modulate, regulate, control and/or inhibit cancer cell virulence and tumor growth. More specifically, the invention provides implantable cell engrafted biocompatible matrices that can modulate cancer cell virulence (e.g., proliferation, metastasis, invasiveness). The teachings presented below provide sufficient guidance to make and use the materials and methods of the present invention, and further provide sufficient guidance to identify suitable criteria and subjects for testing, measuring, and monitoring the performance of the materials and methods of the present invention.

Cancer virulence: Most cancers share six common features, namely self-sufficient growth, insensitivity to antigrowth signals, tumor invasion and metastasis, limitless replicative potential, sustained angiogenesis, and evasion of apoptosis. Several common molecular pathways tend to be dysregulated in cancer cells. Two of these pathways involve the p53 and the pRb transcription factors, which are commonly referred to as “tumor suppressors” since their inactivation promotes cancer development. The p53 pathway integrates cellular information regarding DNA damage and oxidative stress to implement decisions about slowing cell cycle progression or entering apoptosis. The pRb pathway regulates cellular proliferation by controlling passage through the cell cycle. Derangement of these pathways allows cancer cells to ignore physiological stresses and bypass normal cellular checkpoints in order to proliferate supra-physiologically.

Other genes that are frequently dysregulated in cancers include, for example, hypoxia-inducible factor 1-alpha (HIF-1α), receptor tyrosine kinases (RTKs, including many growth factor receptors) and phosphoinositol-3-kinase (PI3K), nuclear factor kappa B (NF-κB), and SMADs (involved in the TGF-β pathway). The temporal order of gene dysregulation is also important in cancer development. In addition, the early activation of telomerase (hTERT) allows developing cancer cells to divide limitlessly and avoid entering replicative senescence.

Emerging evidence indicates that a small subset of cancer cells—cancer stem cells (CSC)—is the major tumor sustaining cell type. Cancer stem cells accumulate tumorigenic mutations and can generate heterogeneous tumors from a single cell. Experimental evidence for cancer stem cells includes the observation that only a small fraction of solid tumor cells in most cancers are clonogenic in vitro and can form heterogeneous tumors in vivo. These cell subpopulations are functionally distinct and display different sets of molecular markers. Changes in cancer stem cells markers can therefore be used as indicators of changes in overall cancer virulence.

Cancer stem cells can reside within a specialized hypoxic niche. Thus, leaky tumor blood vessels can encourage tumor virulence by promoting intratumoral hypoxia to stimulate cancer stem cell proliferation and virulence. Furthermore, brain cancer stem cells tend to reside in intimate contact with tumor vasculature. The cancer stem cell paradigm yields other implications for cancer research and treatment. For example, cancer stem cells are more resistant to traditional pharmacotherapy due to lack of perfusion access, relatively low proliferation rates, and overexpression of drug efflux transporters. In addition, cancer stem cells themselves can invade and metastasize, and cancer stem cells and metastasizing cells share many properties.

Tumor vasculature: Angiogenesis is essential for the development of pathologic tissues such as cancer. Generally, there is a tight balance between pro-angiogenic and anti-angiogenic factors that maintains vascular and tissue homeostasis. Many pro-angiogenesis factors have been identified, including the VEGF and FGF families, and many endogenous angiogenesis inhibitors have been identified, including extracellular matrix fragments (e.g., endostatin, a fragment of collagen XVIII) and other circulating molecules (e.g., thrombospondin). Without angiogenic microvasculature, tumors are unable to grow to more than about 1 mm³ in volume, thereby remaining dormant and generally benign. However, once a tumor undergoes the “angiogenic switch” (which, for example, can be caused by p53 dysfunction), new vessels are recruited, thereby increasing tumor microvascular density and allowing the tumor to grow and become aggressive. To build new vessels, tumor vessel endothelial cells are recruited from circulation (from circulating mature or progenitor endothelial cells) or sprout from existing vessels.

Tumor vessels, which are comprised mainly of endothelial cells, possess abnormal architecture, which results in high permeability. High vessel permeability contributes to intratumoral hypoxia and acidosis, and elevated interstitial pressure, which can facilitate the outward spread of cancers and impede soluble molecule entry into the tumor. In addition, hypoxia contributes to tumor virulence, in part through cancer stem cell stimulation. Tumor endothelial cells obtain a dysregulated phenotype via an imbalance of pro- and anti-angiogenic factors. Tumor-derived nitric oxide (NO) also contributes to the endothelial cell dysfunction and disorganization seen in tumor vessels. Furthermore, “normalization” of the tumor vasculature by anti-angiogenesis therapies can restore the balance of pro- and anti-angiogenic factors and partially explains the successes of such therapies. Other endothelial cell abnormalities in tumor vessels include an “activated” integrin expression pattern, dysregulated leukocyte adhesion, abnormal responses to oxidative stress, and abnormal mechanosensing.

Cancer-Stroma Heterotypic Interactions: Even with dysregulated proliferation, cancer cells still respond to environmental cues and heterotypic regulation. Solid tumors contain, in addition to the cancer cells themselves, many types of stromal cells. Paracrine crosstalk between cancer cells and cells of the microenvironment can enhance tumor proliferation, local invasion, and distant metastasis. Therefore it may be that the microenvironment is required to facilitate tumor malignancy. For example, many carcinomas (e.g., “carcinomas in situ”) are bounded by their basement membranes until they recruit appropriate stomal cells to facilitate their escape and further malignant transformation. Two well-studied cell types that contribute to tumor virulence are fibroblasts and macrophages.

Fibroblasts are the predominant non-malignant cell types in most epithelial tumors. These “carcinoma-associated fibroblasts” (CAF) differ from normal tissue fibroblasts in that they are often contractile (myofibroblasts) and secrete collagenases, matrix metalloproteinases (MMPs), extracellular matrix components, and a wide range of growth factors (e.g., HGF, IGF, VEGF, FGF, Wnt) and other factors (e.g., IL-6, SDF-1). Together, these secreted factors directly support carcinoma cells and recruit blood vessels and other cells to tumors. The immune system is similarly co-opted and locally modified by tumors. Immune cells can initially serve as sentinels, but can ultimately be used by cancer cells to circumvent immune recognition and attack. For example, tumor-associated macrophages (TAM) block cytotoxic T cell-mediated actions (via IL-10 secretion), generate free radicals (which can damage DNA, increasing the number of oncogenic mutations of cancers), and modulate NF-κB signaling. Additionally, TAM can recruit blood vessels, remodel the extracellular matrix to facilitate invasion and metastasis, and regulate local inflammation. Conscripted regulatory T cells can also aid cancer virulence by attenuating the overall immune response to cancers.

Many carcinomas acquire the ability to invade and metastasize by undergoing a sustained, reversible phenotypic change from an epithelial phenotype to a mesenchymal phenotype. This “epithelial-mesenchymal transition” (EMT) also allows carcinoma cells to contribute to the myofibroblast pool in the stroma. The EMT is facilitated by a cells' extracellular matrix and humoral environment (e.g., TGF-β, MMP-3) and leads to changes in the expression of cytoskeletal and cell adhesion molecules (e.g., upregulation of Vimentin and N-Cadherin and downregulation of E-Cadherin) in cancer cells which facilitate invasion and metastasis. Several transcription factors (e.g., Snail, Twist, and Slug) play central roles in the EMT. For example, Snail is highly expressed in the invasive front of invasive carcinomas and integrates signals from many growth and differentiation pathways (e.g., RTKs, Wnt, integrins, TGF-β, MAPK, PI3K, and others). After metastasis and passage through vasculature or lymphatics, cancer cells can revert to an epithelial phenotype to colonize new sites. Interestingly, cells that undergo EMT have similar properties as cancer stem cells.

Endothelial cells as paracrine regulators: Endothelial cells constitute the innermost cell layers of both blood vessels and lymphatics and have many unique regulatory roles. These include control of vasomotor tone, thrombosis and hemostasis, vascular permeabiltiy, cell trafficking/migration, and inflammation. Many endothelial cell functions are affected by local biochemical and biomechanical stimuli, and are cell density- and state-dependent. The endothelium is therefore a plastic organ capable of adapting to a variety of physiologic and pathophysiologic situations. In vitro, confluent/quiescent endothelial cells suppress the proliferation of vascular smooth muscle cells (SMC), whereas subconfluent/activated endothelial cells have the opposite effect. Additionally, many endothelial cell secreted products have direct regulatory roles in cancer behavior. For example, endothelins, which are potent endogenous vasodilatory peptides, are associated with breast tumor invasiveness and with prostate cancer bone metastasis, TGF-β can support or suppress cancer cell proliferation, and CTGF is associated with decreased tumor proliferation and invasion.

Endothelial cells can play a role in cancer cell virulence. For example, bone marrow endothelial cells in hematologic malignancies have an activated phenotype. Similarly, the activation of quiescent endothelial cells is important for angiogenic neovascularization and cancer viralence. Blockade of the mTOR and NF-κB pathways causes marked reduction in endothelial cell activation and angiogenic potential, even in the presence of a pro-angiogenic milieu.

In large blood vessels, where endothelium serves as both the epithelium lining the lumen and as the microvasculature that perfuses the vessel wall, perivascular cell engrafted biocompatible matrices can regulate both native endothelial cell regeneration/repair and vascular smooth muscle (mesenchymal) hyperplasia. In other organs, where epithelium is distinct from endothelium, cell endgrafted endothelial cells are expected to exert control mainly over native epithelium.

The phenotype of tumor vessel endothelial cells—including dysregulated responses to oxidative and mechanical stresses, increased permeability, dysregulated leukocyte attachment, and altered mechanosensing compared to endothelial cells of healthy, quiescent vessels—is “dysfunctional” or “activated” similarly to endothelial cells exposed to chronic inflammatory stimuli. Local endothelial dysfunction also precedes atherosclerostic vascular disease (AVD). This concurrence can serve as another manifestation of the link between inflammation and cancer pathogenesis and could explain why both processes, AVD and cancer, involve similar sets of biochemical mediators (e.g., IL-1β and TNF-60 ) and risk factors (family history, smoking). Additionally, dysfunctional tumor endothelium, which is pro-thrombotic, could contribute to the hypercoagulable state associated with cancer. Finally, direct endothelial effects could contribute to the mechanism whereby statins and NSAIDs (anti-inflammatory medications which directly affect endothelial cell health) modulate the risk of developing cancer.

Without wishing to be bound by theory, it is hypothesized that the microvascular endothelial cells of tumors serve as local tumor regulators that, like other stromal cells, are modified by the tumor to support tumor virulence. in addition, the substrate of tumor endothelial cells are diseased, as manifested by “dysfunctional” endothelial cell adhesion molecule expression (e.g., α,β, integrin) and “inflammatory” extracellular matrix (e.g., oncofetal fibronectin) synthesized by tumor endothelial cells. It is further hypothesized that the cell engrafted biocompatible matrices inhibit cancer cell virulence by providing normal, healthy substratum-adherent endothelial cells which can restore epithelial control of local mesenchyme/stroma via paracrine signaling.

Again, without wishing to be bound by theory, it is further hypothesized that the endothelial cells of blood vessels that perfuse organs provide not only conduits for blood and nutrient access and egress but are themselves biosensors and bioregulators. From the privileged site that vessels occupy as they pervade organs, vascular endothelial cells exert paracrine regulation of adjacent cells. It is further hypothesized that the relationship between endothelial cells and their underlying substrata is essential. If either component of the unit is disordered or diseased, tumor virulence is promoted rather than restricted. Endothelial cells therefore inhibit cancer virulence only when endothelial cell adhesion to their substrate is intact, for example, engrafted. Free endothelial cells are immunogenic and endothelial cells or abnormal substrata promote injury rather than repair. Moreover, as noted above, abnormal endothelial cell architecture can promote tumor virulence.

Abnormal cells include, for example, neoplastic cells, hyperplastic cells, cancerous cells, precancerous cells, metastasizing cells, malignant cells, tumor cell, cancer stem cell, progenitor cell, oncogenic cells, invasive cells, abnormal tissues, cells within abnormal tissues, cells susceptible to or undergoing uncontrolled growth or proliferation, mutated cells, whether inherited mutations or spontaneously mutated or the result of infection or carcinogens.

### Implantable Material

General Considerations: The implantable material of the present invention comprises cells engrafted on, in and/or within a biocompatible matrix. Engrafted means securedly attached via cell to cell and/or cell to matrix interactions such that the cells meet the functional or phenotypical criteria set forth herein and withstand the rigors of the preparatory manipulations disclosed herein. As explained elsewhere herein, an operative embodiment of implantable material comprises a population of cells associated with a supporting substratum, preferably a differentiated cell population and/or a near-confluent, confluent or post-confluent cell population, having a preferred functionality and/or phenotype. Examples of preferred configurations suitable for use in this manner are disclosed in U.S. patent application Ser. No. 11/792,350, based on International Patent Application No. PCT/US05/43967, filed on Dec. 6, 2005, the entire contents of each of which are herein incorporated by reference. Related flowable compositions suitable for use in accordance with the present invention are disclosed in U.S. patent application Ser. No. 11/792,284, based on International Patent Application No. PCT/US05/43844, filed on Dec. 6, 2005, the entire contents of each of which are herein incorporated by reference.

Complex substrate specific interactions regulate the intercellular morphology and secretion of the cells and, accordingly, also regulate the functionality and phenotype of the cells associated with the supporting substratum. Cells associated with certain preferred biocompatible matrices, contemplated herein, can grow and conform to the architecture and surface of the local struts of matrix pores with less straining as they mold to the matrix. Also, the individual cells of a population of cells associated with a matrix retain distinct morphology and secretory ability even without complete contiguity between the cells. Further, cells associated with a biocompatible matrix can no exhibit planar restraint, as compared to similar cells grown as a monolayer on a tissue culture plate.

It is understood that embodiments of implantable material likely shed cells during preparatory manipulations and/or that certain cells are not as securely attached as are other cells. All that is required is that implantable material comprises cells associated with a supporting substratum that meet the functional or phenotypical criteria set forth herein.

That is, interaction between the cells and the matrix during the various phases of the cells' growth cycle can influence the cells' phenotype, with the preferred inhibitory phenotype described elsewhere herein correlating with quiescent cells (i.e., cells which are not in an exponential growth cycle). As explained elsewhere herein, it is understood that, while a quiescent cell typifies a population of cells which are near-confluent, confluent or post-confluent, the inhibitory phenotype associated with such a cell can be replicated by manipulating or influencing the interaction between a cell and a matrix so as to render a cell quiescent-like.

The implantable material of the present invention was developed on the principles of tissue engineering and represents a novel approach to addressing the above-described clinical needs. The implantable material of the present invention is unique in that the viable cells engrafted on, in and/or within the biocompatible matrix are able to supply to the cancer site multiple cell-based products in physiological proportions under physiological feed-back control. As described elsewhere herein, the cells suitable for use with the implantable material include endothelial, endothelial-like, non-endothelial cells or analogs thereof. Local delivery of multiple compounds by these cells in a physiologically-dynamic dosing provide more effective regulation of the processes responsible for inhibiting cancer cell virulence and diminishing the clinical sequel associated with cancer and tumorigenesis.

The implantable material of the present invention, when deposited at, near, adjacent, in the vicinity of, or contacted with the surface of a cancer site serves to reestablish homeostasis. That is, the implantable material of the present invention can provide an environment which mimics supportive physiology and is conducive to the management and inhibition of cancer cell virulence and tumor growth.

For purposes of the present invention, contacting means directly or indirectly interacting with an interior or exterior surface or volume of a cancer and/or tumor site as defined elsewhere herein. In the case of certain preferred embodiments, actual physical contact is not required for effectiveness. In other embodiments, actual physical contact is preferred. All that is required to practice the present invention is deposition of the implantable material at, adjacent to, or in the vicinity of a cancer and/or tumor site in an amount effective to treat the cancer and/or tumor. In the case of certain cancers, a cancer and/or tumor site can clinically manifest on an interior anatomical location, for example, on an interior or exterior surface or volume of a tissue or organ. In the case of other cancers, a cancer and/or tumor site can clinically manifest on an exterior surface, for example, a cancer of the epithelial tissue of the skin. In some cancers, a cancer and/or tumor site can clinically manifest on both an interior surface and an exterior surface of the anatomical location. The present invention is effective to treat any of the foregoing clinical manifestations.

For example, endothelial cells can release a wide variety of agents that in combination can inhibit or mitigate adverse physiological conditions associated with cancer virulence and tumorigenesis. As exemplified herein, a composition and method of use that recapitulates normal physiology and dosing is useful to treat, inhibit and manage cancer. Typically, treatment includes placing the implantable material of the present invention at, adjacent to or in the vicinity of the cancer site or tumor. When wrapped, wrapped around, deposited, or otherwise contacting a cancer and/or tumor site, the cells of the implantable material can provide regulatory signaling to the cancer and/or tumor site, for example, within the cancer and/or tumor site. It is also contemplated that, while inside or outside the cancer and/or tumor site, the implantable material of the present invention comprising a biocompatible matrix or particle with engrafted cells provide a continuous supply of multiple regulatory and therapeutic compounds from the engrafted cells to the cancer and/or tumor site.

Cell Source: As described herein, the implantable material of the present invention comprises cells. Cells can be allogeneic, xenogeneic or autologous. In certain embodiments, a source of living cells can be derived from a suitable donor. In certain other embodiments, a source of cells can be derived from a cadaver or from a cell bank.

In one currently preferred embodiment, cells are endothelial cells. Endothelial cells can be obtained from small vessels, or large vessels. In a particularly preferred embodiment, such endothelial cells are obtained from vascular tissue, preferably but not limited to arterial tissue. As exemplified below, one type of vascular endothelial cell suitable for use is an aortic endothelial cell. Another type of vascular endothelial cell suitable for use is umbilical cord venous endothelial cells. And, another type of vascular endothelial cell suitable for use is coronary artery endothelial cells. Yet another type of vascular endothelial cell suitable for use is saphenous vein endothelial cells. Yet other types of vascular endothelial cells suitable for use with the present invention include pulmonary artery endothelial cells and iliac artery endothelial cells. In another currently preferred embodiment, suitable endothelial cells can be obtained from non-vascular tissue. Non-vascular tissue can be derived from any anatomical structure or can be derived from any non-vascular tissue or organ. Exemplary anatomical structures include structures of the vascular system, the renal system, the reproductive system, the genitourinary system, the gastrointestinal system, the pulmonary system, the respiratory system and the ventricular system of the brain and spinal cord.

In another embodiment, endothelial cells can be derived from endothelial progenitor cells, such as early or late endothelial progenitor cells, or stem cells. In some embodiments, the endothelial cells are bone marrow endothelial cells. In other preferred embodiments, cells can be non-endothelial cells that are allogeneic, xenogeneic or autologous and can be derived from vascular, neural or other tissue or organ. Cells can be selected on the basis of their tissue source and/or their immunogenicity. Exemplary non-endothelial cells include epithelial cells, neural cells, secretory cells, smooth muscle cells, fibroblasts, stem cells, endothelial progenitor cells, cardiomyocytes, keratinocytes, secretory and ciliated cells. The present invention also contemplates any of the foregoing which are genetically altered, modified or engineered.

In another currently preferred embodiment, cells are epithelial cells. In a particularly preferred embodiment, such epithelial cells are obtained from gastrointestinal tissue, tracheal-bronchial-pulmonary tissue, genito-urinary tissue, lymphatic tissue and/or glandular tissue, or another epithelial cell source. According to various embodiments, the epithelial cells are squamous cells, cuboidal cells, columnar cells and/or transitional tissue.

In a further embodiment, two or more types of cells are co-cultured to prepare the present composition. For example, a first cell can be introduced into the biocompatible implantable material and cultured until confluent. The first cell type can include, for example, endothelial cells, epithelial cells, neural cells, secretory cells, smooth muscle cells, fibroblasts, stem cells, nerve stem cells, endothelial progenitor cells, keratinocytes, a combination of endothelial cells and keratinocytes, a combination of smooth muscle cells and fibroblasts, any other desired cell type or a combination of desired cell types suitable to create an environment conducive to growth of the second cell type. Once the first cell type has reached confluence, a second cell type is seeded on top of the first confluent cell type in, on or within the biocompatible matrix and cultured until both the first cell type and second cell type have reached confluence. The second cell type can include, for example epithelial cells, neural cells, secretory cells, smooth muscle cells, fibroblasts, stem cells, nerve stem cells, endothelial cells, endothelial progenitor cells, keratinocytes or any other desired cell type or combination of cell types. It is contemplated that the first and second cell types can be introduced step wise, or as a single mixture. It is also contemplated that cell density can be modified to alter the ratio of the first cell type to the second cell type.

To prevent over-proliferation of smooth muscle cells or another cell type prone to excessive proliferation, the culture procedure and timing can be modified. For example, following confluence of the first cell type, the culture can be coated with an attachment factor suitable for the second cell type prior to introduction of the second cell type. Exemplary attachment factors include coating the culture with gelatin to improve attachment of endothelial cells. According to another embodiment, heparin can be added to the culture media during culture of the second cell type to reduce the proliferation of the first cell type and to optimize the desired first cell type to second cell type ratio. For example, after an initial growth of smooth muscle cells, heparin can be administered to control smooth muscle cell growth to achieve a greater ratio of endothelial cells to smooth muscle cells.

In a preferred embodiment, a co-culture is created by first seeding a biocompatible implantable material with smooth muscle cells to create structures, for example, but not limited to, structures that mimic the size and/or shape of the cancer site and/or its surrounding vasculature. Once the smooth muscle cells have reached confluence, endothelial cells, epithelial cells, endothelial-like cells, epithelial-like cells, or non-endothelial cells are seeded on top of the cultured smooth muscle cells on the implantable material to created a completed substrata.

All that is required of the cells of the present composition is that they exhibit one or more preferred phenotypes or functional properties. As described earlier herein, the present invention is based on the discovery that a cell having a readily identifiable phenotype when associated with a preferred matrix (described elsewhere herein) can inhibit, restore and/or otherwise modulate cell physiology and/or homeostasis associated with the treatment of a cancer and/or tumor site generally.

For purpose of the present invention, one such preferred, readily identifiable phenotype typical of cells of the present invention is an ability to inhibit or otherwise interfere with smooth muscle cell proliferation and/or migration. Smooth muscle cell proliferation can be determined using an in vitro smooth muscle cell proliferation assay and smooth muscle cell migration can be determining using an in vitro smooth muscle cell migration assay, both of which are described below. The ability to regulate smooth muscle cell proliferation and/or migration is referred to herein as the inhibitory phenotype.

One other readily identifiable phenotype exhibited by cells of the present composition is that they are able to regulate gibroblast proliferation and/or migration and collagen deposition and/or accumulation. Fibroblast activity and collagen deposition activity can be determined using an in vitro fibroblast proliferation, in vitro fibroblast migration and/or an in vitro collagen accumulation assay, each of which are described below. The ability to tegulate fibroblast proliferation and/or migration is also referred to herein as the inhibitory phenotype.

Another readily identifiable phenotype exhibited by cells of the present composition is that they are anti-thrombotic or are able to inhibit platelet adhesion and aggregation. Anti-thrombotic activity can be determined using an in vitro heparan sulfate assay and/or an in vitro platelet aggregation assay, described below.

An additional readily identifiable phenotype exhibited by cells of the present composition is the ability to inhibit cancer cell proliferation and/or cancer cell invasiveness in vitro. Cancer cell proliferation and/or cancer cell invasiveness can be determined using an in vitro chemoinvasion/chemomigration assay.

A further readily identifiable phenotype exhibited by cells of the presnet composition is the ability to restore the proteolytic balance, the MMP-TIMP balance, the ability to decrease expression of MMPs relative to the expression of TIMPs, or the ability to increase expression of TIMPs relative to the expression of MMPs. proteolytic balance activity can be determined using an in vitra TIMP assay and/or an in vitro MMP assay described below.

In a typical operative embodiment of the present invention, cells need not exhibit more than one of the foregoing phenotypes. In certain embodiments, cells can exhibit more than one of the foregoing phenotypes.

While the foregoing phenotypes each typify a functional endothelial cell, such as but not limited to a vascular endothelial cell, a non-endothelial cell exhibiting such a phenotype(s) is considered endothelial-like for purposes of the present invention and thus suitable for use with the present invention. Cells that are endothelial-like are also referred to herein as functional analogs of endothelial cells; or functional mimics of endothelial cells. Thus, by way of example only, cells suitable for use with the materials and methods disclosed herein also include epithelial cells, stem cells or progenitor cells that give rise to endothelial-like or epithelial-like cells; cells that are non-endothelial or non-epithelial cells in origin yet perform functionally like an endothelial or epithelial cell, respectively, using the parameters set forth herein; cells of any origin which are engineered or otherwise modified to have endothelial-like or epithelial-like functionality using the parameters set forth herein.

Typically, cells of the present invention exhibit one or more of the aforementioned functionalities and/or phenotypes when present and associated with a supporting substratum, such as the biocompatible matrices described herein. It is understood that individual cells attached to a matrix and/or interacting with a specific supporting substratum exhibit an altered expression of functional molecules, resulting in a preferred functionality or phenotype when the cells are associated with a matrix or supporting substratum that is absent when the cells are not associated with a supporting substratum.

According to one embodiment, the cells exhibit a preferred phenotype when the basal layer of the cell is associated with a supporting substratum. According to another embodiment, the cells exhibit a preferred phenotype when present in confluent, near confluent or post-confluent populations. As will be appreciated by one of ordinary skill in the art, populations of cells, for example, substrate adherent cells, and confluent, near confluent and post-confluent populations of cells, are identifiable readily by a variety of techniques, the most common and widely accepted of which is direct microscopic examination. Others include evaluation of cell number per surface area using standard cell counting techniques such as but not limited to a hemacytometer or coulter counter.

Additionally, for purposes of the present invention, endothelial-like cells include but are not limited to cells which emulate or mimic functionally and phenotypically the preferred populations of cells set forth herein, including, for example, differentiated endothelial cells and confluent, near confluent or post-confluent endothelial cells, as measured by the parameters set forth herein.

Thus, using the detailed description and guidance set forth below, the practitioner of ordinary skill in the art will appreciate how to make, use, test and identify operative embodiments of the implantable material disclosed herein. That is, the teachings provided herein disclose all that is necessary to make and use the present invention's implantable materials. And further, the teachings provided herein disclose all that is necessary to identify, make and use operatively equivalent cell-containing compositions. At bottom, all that is required is that equivalent cell-containing compositions are effective to treat, manage, modulate and/or ameliorate a cancer site in accordance with the methods disclosed herein. As will be appreciated by the skilled practitioner, equivalent embodiments of the present composition can be identified using only routine experimentation together with the teachings provided herein.

In certain preferred embodiments, endothelial cells used in the implantable material of the present invention are isolated from the aorta of human cadaver donors. Each lot of cells is derived from a single donor or from multiple donors, tested extensively for endothelial cell purity, biological function, the presence of bacteria, fungi, human pathogents and other adventitious agents. The cells are cryopreserved and banked using well-known techniques for later expansion in culture for subsequent formulation in biocompatible implantable materials.

Examples of preferred configurations suitable for use in this manner are disclosed in U.S. patent application Ser. No. 11/792/350, based on International Patent Application No. PCT/US05/43967, filed on Dec. 6, 2005, the entire contents of each of which are herein incorporated by reference. Related flowable compositions suitable for use in accordance with the present invention are disclosed in U.S. patent application Ser. No. 11/792,284, based on International Patent Application No. PCT/US05/43844, filed on Dec. 6, 2005, the entire contents of each of which are herein incorporated by reference.

Cell Preparation: As stated above, suitable cells can be obtained from a variety of tissue types and cell types. In certain preferred embodiments, human aortic endothelial cells used in the implantable material are isolated from the aorta of cadaver donors by collagenase digestion. In other embodiments, porcine aortic endothelial cells are isolated from normal porcine aorta by a similar procedure used to isolate human aortic endothelial cells. Each lot of cells can be derived from a single donor or from multiple donors, tested extensively for endothelial cell viability, purity, biological function, the presence of mycoplasma, bacteria, fungi, yeast, human pathogens and other adventitious agents. The cells are further expanded, characterized and cryopreserved to form a working cell bank at the third to sixth passage using well-known techniques for later expansion in culture and for subsequent formulation in biocompatible implantable material.

In some embodiments, cells of the invention can be cultured to a particular growth stage or cell density before being engrafted onto a biocompatible matrix. For example, cells, such as isolated endothelial cells, can be subconfluent and activated, confluent and quiescent, and the like when engrafted.

The human or porcine aortic endothelial cells are prepared in T-75 flasks or 10-cm dishes pre-treated by the addition of approximately 15 ml of endothelial cell growth media per flask. Alternatively flasks/dishes are pretreated for ˜30 minutes with 0.1% gelatin solution (˜1 mL per 5 cm² area), after which the gelatin solution is aspirated shortly before adding cells and media. Human aortic endothelial cells are prepared in endothelial Growth Media (EGM-2, Lonza Biosciences, Basel, Switzerland). EGM-2 consists of Endothelial Cell Basal Media (EBM-2, Lonza Biosciences, Basel, Switzerland) supplemented with EGM-2singlequots, which contain 2% FBS; an additional 3-7% FBS can be added to the media to make a final concentration of 5-10% FBS by volume. Porcine cells are prepared in EBM-2 supplemented with 5% FBS and 50 μg/ml gentamicin. The flasks are placed in an incubator maintained at approximately 37° C. and 5% CO₂/95% air, 90% humidity for a minimum of 30 minutes. One or two vials of the cells are removed from the −160° C. to −140° C. freezer and thawed at approximately 37° C. Each vial of thawed cells is seeded into two T-75 flasks at a density of approximately 3×10³ cells per cm², preferably, but no less than 1.0×10³ and no more than 7.0×10³; and the flasks containing the cells are returned to the incubator. After about 8-24 hours, the spent media is removed and replaced with fresh media. The media is changed every two to three days, thereafter, until the cells reach approximately 85-100% cibfkyebce preferably, but no less than 60% and no more than 100%. When the implantable material is intended for clinical application, only antibiotic-free media is used in the post-thaw culture of human aortic endothelial cells and manufacture of the implantable material of the present invention.

The endothelial cell growth media is then removed, and the monoplayer of cells is rinsed with 10 ml of HEPES buffered saline (HEPES) or phosphate-buferred saline (PBS). The HEPES (PBS) is removed, and 3 ml of trypsin is added to detach the cells from the surface of the T-75 flask (or 2 mL for a 10-cm dish). Once detachment has occurred, 2 (or 2) ml of trypsin neutralizing solution (TNS) is added to stop the enzymatic reaction. An additional 5 ml of HEPES is added, and the cells are emumerated using a hemocytometer. The cell suspension is centrifuged and adjusted to a density of, in the case of human cells, approximately 2.0-1.75×10⁶ cells/ml using EGM-2 without antibiotics, or in the case of porcine cells, approximately 2.0-1.50×10⁶ cells/ml using EBM-2 supplemented with 5% FBS and 50 μg/ml gentamicin.

Biocompatible Matrix: According to the present invention, the implantable material comprises a biocompatible matrix. The matrix is permissive for cell growth and attachment to, on or within the matrix. The matrix is flexible and conformable. The matrix can be a solid, a semi-solid or flowable porous composition. For purposes of the present invention, flowable composition means a composition susceptible to administration using an injection or injection-type delivery device such as, but not limited to, a needle, a syringe or a catheter. Other delivery devices which employ extrusion, ejection or expulsion are also contemplated herein. Porous matrices are preferred. The matrix also can be in the form of a flexible planar form. The matrix also can be in the form of a gel, a foam, a suspension, a particle, a microcarrier, a macrocarrier, a microcapsule, or a fibrous structure. A preferred flowable composition is shape-retaining. A currently preferred matrix has a particulate form. The biocompatible matrix can comprise particles and/or microcarriers and/or macrocarriers and the particles and/or microcarriers and/or macrocarriers can further comprise gelatin, collagen, fibronectin, fibrin, laminin or an attachment peptide. One exemplary attachment peptide is a peptide of sequence arginine-glycine-aspartate (RGD).

The matrix, when implanted on a surface of a cancer and/or tumor site, can reside at the implantation site for at least about 7-90 days, preferably about at least 7-14 days, more preferably about at least 14-28 days, most preferably about at least 28-90 days before it bioerodes.

One preferred matrix is Gelfoam® (Pfizer, Inc., New York, N.Y.), an absorbable gelatin sponge (hereinafter “Gelfoam® matrix”). Another preferred matrix is Surgifoam® (Johnson & Johnson, New Brunswick, N.J.), also an absorbable gelatin sponge. Gelfoam® and Surgifoam® matrices are porous and flexible surgical sponges prepared from a specially treated, purified porcine dermal gelatin solution.

According to another embodiment, the biocompatible matrix material can be a modified matrix material. Modifications to the matrix material can be selected to optimize and/or to control function of the cells, including the cells' phenotype (e.g., the inhibitory phenotype) as described above, when the cells are associated with the matrix. According to one embodiment, modifications to the matrix material include coating the matrix with attachment factors or adhesion peptides that enhance the ability of the cells to regulate smooth muscle cell and/or fibroblast proliferation and migration, to increase TIMP production, to optimize the proteolytic balance (the MMP/TIMP balance), to decrease inflammation, to increase heparan sulfate production, to increase prostacyclin production, and/or to increase FGF2, TGF-β, and nitric oxide (NO) production.

According to some embodiments, the properties of the matrix itself are altered. For example, the elastic modulus, plasticity, and/or stiffness of a matrix material such as, for example, GELFOAM® can be altered to maximize paracrine regulatory effects of engrafted cells. The matrix material can be stiffed by, for example, crosslinking the matrix material with a chemical agent such as EDAC (1-Ethyl-3-[3-dimethylaminopropyl]carbodiimide hydrochloride, EMD Biosciences, Gibbstown, N.J.) and/or NHS (amine-reactive succinimidyl ester, Pierce, Rockford, Ill.) The stiffness of the matrix material can be reduced by, for example, autoclaving the matrix material to reduce the number of crosslinks.

According to another embodiment, the matrix is a matrix other than Gelfoam®. Additional exemplary matrix materials include, for example, fibrin gel, alginate, gelatin bead microcarriers, polystyrene sodium sulfonate microcarriers, collagen coated dextran microcarriers, PLA/PGA and pHEMA/MMA copolymers (with polymer ratios ranging from 1-100% for each copolymer). According to one embodiment, a synthetic matrix material, for example, PLA/PGA, is treated with NaOH to increase the hydrophilicity of the material and, therefore, the ability of the cells to attach to the material. According to a preferred embodiment, these additional matrices are modified to include attachment factors or adhesion peptides, as recited and described above. Exemplary attachment factors include, for example, gelatin, collagen, fibronectin, fibrin gel, and covalently attached cell adhesion ligands (including for example RGD) utilizing standard aqueous carbodiimide chemistry. Additional cell adhesion ligands (include peptides having cell adhesion recognition sequences, including but not limited to: RGDY, REDVY, GRGDF, GPDSGR, GRGDY and REDV.

That is, these types of modifications or alterations of a substrate influence the interaction between a cell and a matrix which, in turn, can mediate expression of the preferred inhibitory phenotype described elsewhere herein. It is contemplated that these types of modifications or alterations of a substrate can result in enhanced expression of an inhibitory phenotype; can result in prolonged or further sustained expression of an inhibitory phenotype; and/or can confer such a phenotype on a cell which otherwise in its natural state does not exhibit such a phenotype as in, for example but not limited to, an exponentially growing or non-quiescent cell. Moreover, in certain circumstances, it is preferable to prepare an implantable material of the present invention which comprises non-quiescent cells provided that the implantable material has an inhibitory phenotype in accordance with the requirements set forth elsewhere herein. As already explained, the source of cells, the origin of cells and/or types of cells useful with the present invention are not limited; all that is required is that the cells express an inhibitory phenotype.

Embodiments of Implantable Materials: As stated earlier, implantable material of the present invention can be a flexible planar form or a flowable composition. When in a flexible planar form, it can assume a variety of shapes and sizes, preferably a shape and size which conforms to a contoured surface of a cancer and/or tumor site when situated at or adjacent to or in the vicinity of a cancer and/or tumor site. Examples of preferred configurations suitable for use in this manner are disclosed in U.S. patent application Ser. No. 11/792,350, based on International Patent Application No. PCT/US05/43967, filed on Dec. 6, 2005, the entire contents of each of which are herein incorporated by reference.

Flowable Composition: In certain embodiments contemplated herein, the implantable material of the present invention is a flowable composition comprising a particulate biocompatible matrix which can be in the form of a gel, a foam, a suspension, a particle, a microcarrier, a macrocarrier, a microcapsule, macroporous beads, or other flowable material. The current invention contemplates any flowable composition that can be administered with an injection-type delivery device. For example, a delivery device such as a percutaneous injection-type delivery device is suitable for this purpose as described below. The flowable composition is preferably a shape-retaining composition. Thus, an implantable material comprising cells in, on or within a flowable-type particulate matrix as contemplated herein can be formulated for use with any injectable delivery device ranging in internal diameter from about 18 gauge to about 30 gauge and capable of delivering about 50 mg of flowable composition comprising particulate material containing preferably about 1 million cells in about 1 to about 3 ml of flowable composition.

According to a currently preferred embodiment, the flowable composition comprises a biocompatible particulate matrix such as Gelfoam® particles, Gelfoam® powder, or pulverized Gelfoam® (Pfizer Inc., New York, N.Y.) (hereinafter “Gelfoam particles”), a product derived from porcine dermal gelatin. According to another embodiment, the particulate matrix is Surgifoam™ (Johnson & Johnson, New Brunswick, N.J.) particles, comprised of absorbable gelatin powder. According to another embodiment, the particulate matrix is Cytodex-3 (Amersham Biosciences, Piscataway, N.J.) microcarriers, comprised of denatured collagen coupled to a matrix of cross-linked dextran. According to a further embodiment, the particulate matrix is CultiSper-G (Percell Biolytica AB, Astorp, Sweden) microcarrier, comprised of porcine gelatin. According to another embodiment, the particulate matrix is a macroporous material. According to one embodiment, the macroporous particulate matrix is CytoPore (Amersham Biosciences, Piscataway, N.J.) macrocarrier, comprised of cross-linked cellulose which is substituted with positively charged N,N,-diethylaminoethyl groups.

According to alternative embodiments, the biocompatible implantable particulate matrix is a modified biocompatible matrix. Modifications include those described above for an implantable matrix material.

Related flowable compositions suitable for use in accordance with the present invention are disclosed in U.S. patent application Ser. No. 11/792,284, based on International Patent Application No. PCT/US05/43844, filed on Dec. 6, 2005, the entire contents of each of which are herein incorporated by reference.

Preparation of Implantable Material: Prior to cell seeding, the biocompatible matrix is re-hydrated by the addition of water, buffers and/or culture media such as EGM-2 at approximately 37° C. and 5% CO₂/95% air for 12 to 24 hours. The implantable material is then removed from their re-hydration containers and placed in individual tissue culture dishes. The biocompatible matrix is seeded at a preferred density of approximately 1.5-2.0×10⁵ cells (1.25-1.66×10⁵ cells/cm³ of matrix) and placed in an incubator maintained at approximately 37° C. and 5% CO₂/95% air, 90% humidity for 3-4 hours to 24 hours to facilitate cell attachment. The seeded matrix is then placed into individual containers (Evergreen, Los Angeles, Calif.) or tubes, each fitted with a cap containing a 0.2 μm filter with EGM-2 and incubated at approximately 37° C. and 5% CO₂/95% air. Alternatively, 3 seeded matrices can be placed into 150 mL bottle. The media is changed every two to three days, thereafter, until the cells have reached near-confluence, confluence or post-confluence. The cells in one preferred embodiment are preferably passage 6, but cells of fewer or more passages can be used.

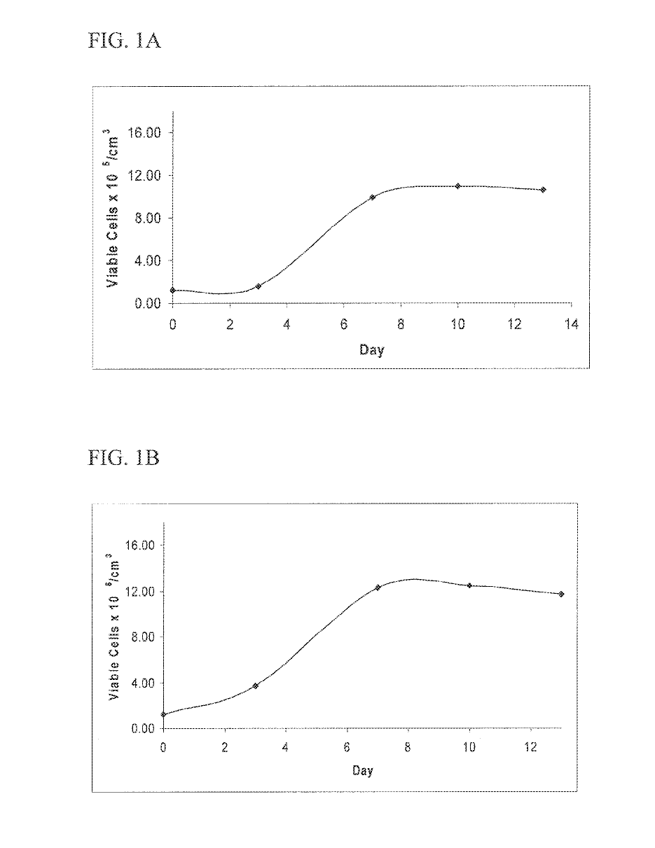

Cell Growth Curve and Confluence: A sample of implantable material is removed on or around days 3 or 4, 6 or 7, 9 or 10, and 12 or 13, the cells are counted and assessed for viability, and a growth curve is constructed and evaluated in order to assess the growth characteristics and to determine whether confluence, near confluence or post-confluence has been achieved. Representative growth curves from two preparations of implantable material comprising porcine aortic endothelial cell implanted lots are presented in FIGS. 1A and 1B. In these examples, the implantable material is in a flexible planar form. Generally, one of ordinary skill will appreciate the indicia of acceptable cell growth at early, mid- and late time points, such as observation of an increase in cell number at the early time points (when referring to FIG. 1A, between about days 2-6), followed by a near confluent phase (when referring to FIG. 1A, between about days 6-8), followed by a plateau in cell number once the cells have reached confluence as indicated by a relatively constant cell number (when referring to FIG. 1A, between about days 8-10) and maintenance of the cell number when the cells are post-confluent (when referring to FIG. 1A, between about days 10-14). For purposes of the present invention, cell populations which are in a plateau for at least 72 hours are preferred.

Cell counts are achieved by complete digestion of the aliquot of implantable material such as with a solution of 0.5 mg/ml collagenase in a CaCl₂solution in the case of gelatin-based matrix materials. After measuring the volume of the digested implantable material, a known volume of the cell suspension is diluted with 0.4% trypan blue (4:1 cells to trypan blue) and viability assessed by trypan blue exclusion. Viable, non-viable and total cells are enumerated using a hemacytometer. Growth curves are constructed by plotting the number of viable cells versus the number of days in culture. Cells are shipped and implanted after reaching confluence.

For purposes of the present invention, confluence is defined as the presence of at least about 4×10⁵ cells/cm³ when in a flexible planar form of the implantable material (1.0×4.0×0.3 cm), and preferable about 7×10⁵ to 1×10⁶ total cells per aliquot (50-70 mg) when in a flowable composition. For both, cell viability is at least about 90% preferably but no less than 80%. If the cells are not confluent by day 12 or 13, the media is changed, and incubation is continued for an additional day. This process is continued until confluence is achieved or until 14 days post-seeding. On day 14, if the cells are not confluent, the lot is discarded. If the cells are determined to be confluent after performing in-process checks, a final media change is performed. This final media change is performed using EGM-2 without phenol red and without antibiotics. Immediately following the media change, the tubes are fitted with sterile plug seal caps for shipping.

The total cell load per human patient will be preferably approximately 1.6-2.6×10⁴ cells per kg body weight, but no less than about 2×10³ and no more than about 2×10⁶ cells per kg body weight.

Evaluation of Functionality and Phenotype: For purposes of the invention described herein, the implantable material is further tested for indicia of functionality and phenotype prior to implantation. For example, conditioned media are collected during the culture period to ascertain levels of heparan sulfate, transforming growth factor-β₁ (TGF-β₁), fibroblast growth factor 2 (FGF2), tissue inhibitors of matrix metalloproteinases (TIMP), and nitric oxide which are produced by the cultured endothelial cells. In certain preferred embodiments, the implantable material can be used for the purposes described herein when total cell number is at least about 2, preferably at least about 4×10⁵ cells/cm³ of implantable material; percentage of viable cells is at least about 80-90%, preferably ≧90%, most preferably at least about 90%; heparan sulfate in conditioned media is at least about 0.23-1.0, preferably at least about 0.5 microg/mL/day; TGF-β₁ in conditioned media is at least about 200-300 picog/mL/day, preferably at least about 300 picog/ml/day; FGF2 in conditioned media is below about 200 picog/ml, preferably no more than about 400 picog/ml; TIMP-2 in conditioned media is at least about 5.0-10.0 ng/mL/day, preferably at least about 8.0 ng/mL/day; NO in conditioned media is at least about 0.5-3.0 μmol/L/day, preferably at least about 2.0 μmol/L/day.

Heparan sulfate levels can be quantified using a routine dimethylmethylene blue-chondroitinase ABC digestion spectrophotometric assay. Total sulfated glycosaminoglycan (GAG) levels are determined using a dimethylmethylene blue (DMB) dye binding assay in which unknown samples are compared to a standard curve generated using known quantities of purified chondroitin sulfate diluted in collection media. Additional samples of conditioned media are mixed with chondroitinase ABC to digest chondroitin and dermatan sulfates prior to the addition of the DMB color reagent. All absorbances are determined at the maximum wavelength absorbance of the DMB dye mixed with the GAG standard, generally around 515-525 nm. The concentration of heparan sulfate per day is calculated by multiplying the percentage heparan sulfate calculated by enzymatic digestion by the total sulfated glycosaminoglycan concentration in conditioned media samples. Chondroitinase ABC activity is confirmed by digesting a sample of purified 100% chondroitin sulfate and a 50/50 mixture of purified heparan sulfate and chondroitin sulfate. Conditioned medium samples are corrected appropriately if less than 100% of the purified chondroitin sulfate is digested. Heparan sulfate levels can also be quantitated using an ELISA assay employing monoclonal antibodies.

TGF-β₁, TIMP, and FGF2 levels can be quantified using an ELISA assay employing monoclonal or polyclonal antibodies, preferably polyclonal. Control collection media can also be quantitated using an ELISA assay and the samples corrected appropriately for TGF-β₁, TIMP, and FGF2 levels present in control media.

Nitric oxide (NO) levels can be quantified using a standard Griess Reaction assay. The transient and volatile nature of nitric oxide makes it unsuitable for most detection methods. However, two stable breakdown products of nitric oxide, nitrate (NO₃) and nitrite (NO₂), can be detected using routing photometric methods. The Griess Reaction assay enzymatically converts nitrate to nitrite in the presence of nitrate reductase. Nitrite is detected colorimetrically as a colored azo dye product, absorbing visible light in the range of about 540 nm. The level of nitric oxide present in the system is determined by converting all nitrate into nitrite, determining the total concentration of nitrite in the unknown samples, and then comparing the resulting concentration of nitrite to a standard curve generated using known quantities of nitrate converted to nitrite.

The earlier-described preferred inhibitory phenotype is assessed using the quantitative heparan sulfate, TGF-β₁, TIMP, NO and/or FGF2 assay described above, as well as quantitative in vitro assays of smooth muscle cell proliferation and migration, fibroblast proliferation, migration and collagen deposition activity, keratinocyte proliferation and migration, and inhibition of thrombosis as follows. For purposes of the present invention, implantable material is ready for implantation when one or more of these alternative in vitro assays confirm that the implantable material is exhibiting the preferred inhibitory phenotype.

To evaluate inhibition of thrombosis in vitro, the level of heparan sulfate associated with the cultured endothelial cells is determined. Heparan sulfate has both anti-proliverative and anti-thrombotic properties. Using either the routine dimethylmethylene blue-chondroitinase ABC digestion spectrophotometric assay or an ELISA assay, both assays are described in detail above, the concentration of heparan sulfate is calculated. The implantable material can be used for the purposes described herein when the heparan sulfate in the conditioned media is at least about 0.23-1.0, preferably at least about 0.5 microg/mL/day.

Another method to evaluate inhibition of thrombosis involves determining the magnitude of inhibition of platelet aggregation in vitro associated with platelet rich-plasma or platelet concentrate (Research Blood Components, Brighton, Mass.). Conditioned media is prepared from post-confluent endothelial cell cultures and added to aliquots of the platelet concentrate. A platelet aggregating agent (agonist) is added to the platelets seeded into 96 well plates as control. Platelet agonists commonly include arachidonate, ADP, collagen type I, epinephrine, thrombin (Sigma-Aldrich Co., St. Louis, Mo.) or ristocetin (available from Sigma-Aldrich Co., St. Louis, Mo.). An additional well of platelets has no platelet agonist or conditioned media added, to assess for baseline spontaneous platelet aggregation. A positive control for inhibition of platelet aggregation is also included in each assay. Exemplary positive controls include aspirin, heparin, indomethacin (Sigma-Aldrich Co., St. Louis, Mo.), abciximab (ReoPro®, Eli Lilly, Indianapolis, Ind.), tirofiban (Aggrastat®, Merck & Co., Inc., Whitehouse Station, N.J.) or eptifibatide (Integrilin®, Millennium Pharmaceuticals, Inc., Cambridge, Mass.). The resulting platelet aggregation of all test conditions are then measured using a plate reader and the absorbance read at 405 nm. The platelet reader measures platelet aggregation by monitoring optical density. As platelets aggregate, more light can pass through the specimen. The platelet reader reports results in absorbance, a function of the rate at which platelets aggregate. Aggregation is assessed as maximal aggregation before the addition of conditioned medium with that after exposure of platelet concentrate to conditioned medium, and to the positive control. Results are expressed as a percentage of the baseline. The magnitude of inhibition associated with the conditioned media samples are compared to the magnitude of inhibition associated with the positive control. According to a preferred embodiment, the implantable material is considered regulatory if the conditioned media inhibits thrombosis by at least about 20% of the control, more preferably by at least about 40% of the control, and most preferably by at least about 60% of the control.

When ready for implantation, the planar form of implantable material is supplied in final product containers, each preferably containing a 1×4×0.3 cm (1.2 cm³), sterile implantable material with preferably approximately 5-8×10⁵ or preferably at least about 4×10⁵ cells/cm³, and at least about 90% viable cells (for example, human aortic endothelial cells derived from a single cadaver donor) per cubic centimeter implantable material in approximately 45-60 ml, preferably about 50 ml, endothelial growth medium (for example, endothelial growth medium (EGM-2), containing no phenol red and no antibiotics). When porcine aortic endothelial cells are used, the growth medium is also EMB-2 containing no phenol red, but supplemented with 5% FBS and 50 μg/ml gentamicin.

In other preferred embodiments, the flowable composition (for example, a particulate form biocompatible matrix) is supplied in final product containers, including, for example, sealed tissue culture containers modified with filter caps or pre-loaded syringes, each preferably containing about 50-60 mg of flowable composition comprising about 7×10⁵ to about 1×10⁶ total endothelial cells in about 45-60 ml, preferably about 50 ml, growth medium per aliquot.

Administration of Implantable Material: When administered in its flowable configuration, the implantable material of the present invention comprises a particulate biocompatible matrix and cells, preferably endothelial cells, more preferably vascular endothelial cells, which are about 90% viable at a preferred density of about 0.8×10⁴ cells/mg, more preferred of about 1.5×10⁴ cells/mg, most preferred of about 2×10⁴ cells/mg, and which can produce conditioned media containing heparan sulfate at least about 0.23-1.0, preferably at least about 0.5 microg/mL/day, TGF-β₁ at at least about 200-300 picog/ml/day, preferably at least about 300 picog/ml/day, and FGF2 below about 200 picog/ml and preferably no more than about 400 picog/ml; TIMP-2 in conditioned media is at least about 5.0-10.0 ng/mL/day, preferably at least about 8.0 ng/mL/day; NO in conditioned media is at least about 0.5-3.0 μmol/L/day, preferably at least about 2.0 μmol/L/day; and, display the earlier-described inhibitory phenotype.

Examples of preferred configurations suitable for use in this manner are disclosed in U.S. patent application Ser. No. 11/792,350, based on International Patent Application No. PCT/US05/43967, filed on Dec. 6, 2005, the entire contents of each of which are herein incorporated by reference. Related flowable compositions suitable for use in accordance with the present invention are disclosed in U.S. patent application Ser. No. 11/792,284, based on International Patent Application No. PCT/US05/43844, filed on Dec. 6, 2005, the entire contents of each of which are herein incorporated by reference.

For purposes of the present invention generally, administration of the implantable material is localized to a site near, in the vicinity of, adjacent or at a cancer and/or tumor site. The site of deposition of the implantable material can also be remote from the cancer and/or tumor site. As contemplated herein, localized deposition can be accomplished as follows.

In a particularly preferred embodiment, the flowable composition is administered percutaneously, entering the patient's body at a suitable location followed by deposition at, adjacent, near, in the vicinity of or in contact with the caner and/or tumor site or the stroma or an interstitial site adjacent to or surrounding the cancer and/or tumor site; delivery and deposition is accomplished using a suitable needle, catheter or other suitable percutaneous delivery device. Alternatively, the flowable composition is delivered percutaneously using a needle, catheter or other suitable delivery device in conjunction with an identifying step to facilitate delivery to a desired site. The identifying step can be accomplished using physical examination, ultrasound, and/or CT scan, to name but a few. The identifying step is optionally performed and not required to practice the methods of the present invention.

Preferably, the implantable material is deposited near a cancer and/or tumor site, either at the cancer and/or tumor site to be treated, or adjacent to or in the vicinity of the caner and/or tumor site. The composition can be deposited in a variety of locations relative to a cancer and/or tumor site. According to a preferred embodiment, an adjacent site is within about 0 mm to 20 mm of the cancer and/or tumor site. In another preferred embodiment, a site is within about 21 mm to 40 mm; in yet another preferred embodiment, a site is within about 41 mm to 60 mm. In another preferred embodiment, a site is within about 61 mm to 100 mm. Alternatively, an adjacent site is any other clinician-determined adjacent location where the deposited composition is capable of exhibiting a desired effect on a cancer and/or tumor site in the proximity of the cancer and/or tumor site. The implantable material need only be implanted in an amount effective to treat a cancer and/or tumor site.

In another embodiment, the implantable material is delivered directly to a surgically-exposed site within a patient's body at, adjacent to or in the vicinity of a cancer and/or tumor site. Also in this case, delivery can be aided by coincident use of an identifying step as described above. Again, the identifying step is optional.

According to another embodiment of the invention, the flexible planar form of the implantable material is delivered locally to a site within the patient's body at or near the cancer and/or tumor site or at a surgically-exposed cancer and/or tumor site or interior cavity at, adjacent to or in the vicinity of a cancer and/or tumor site. In one case, at least one piece of the implantable material is applied to a desired site by applying the implantable material at or around the cancer and/or tumor site. The implantable material need only be implanted in an amount effective to treat a cancer and/or tumor site.

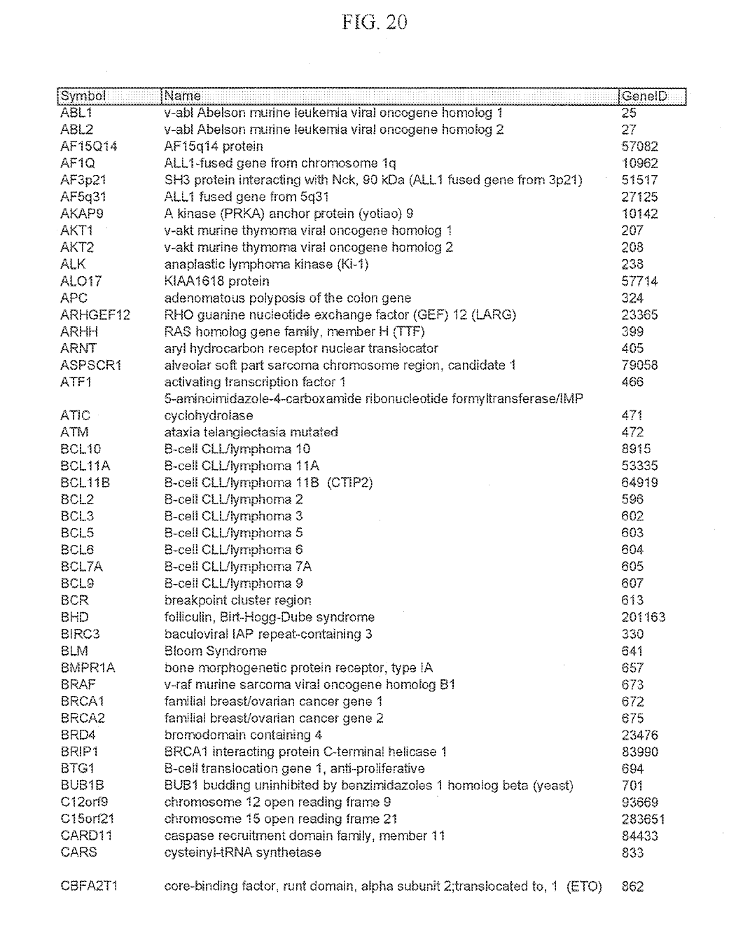

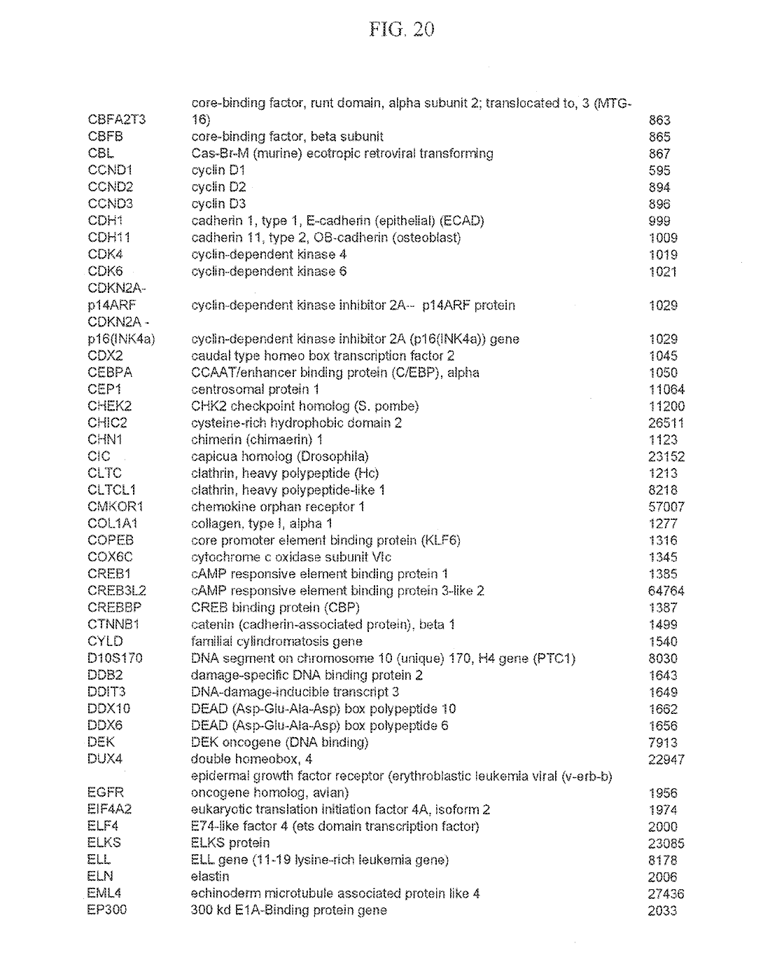

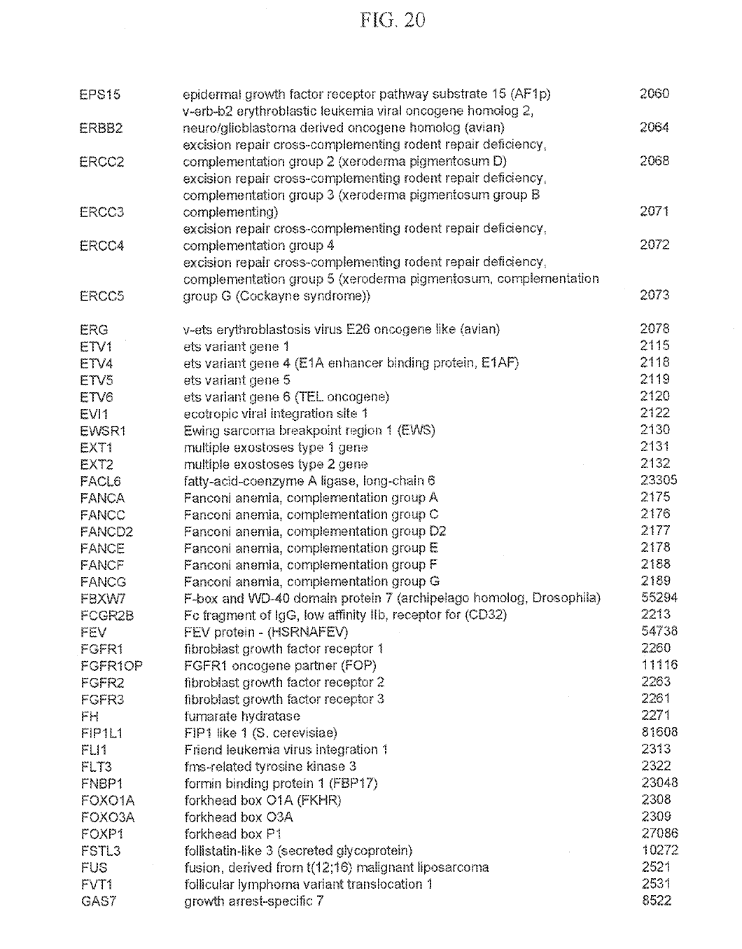

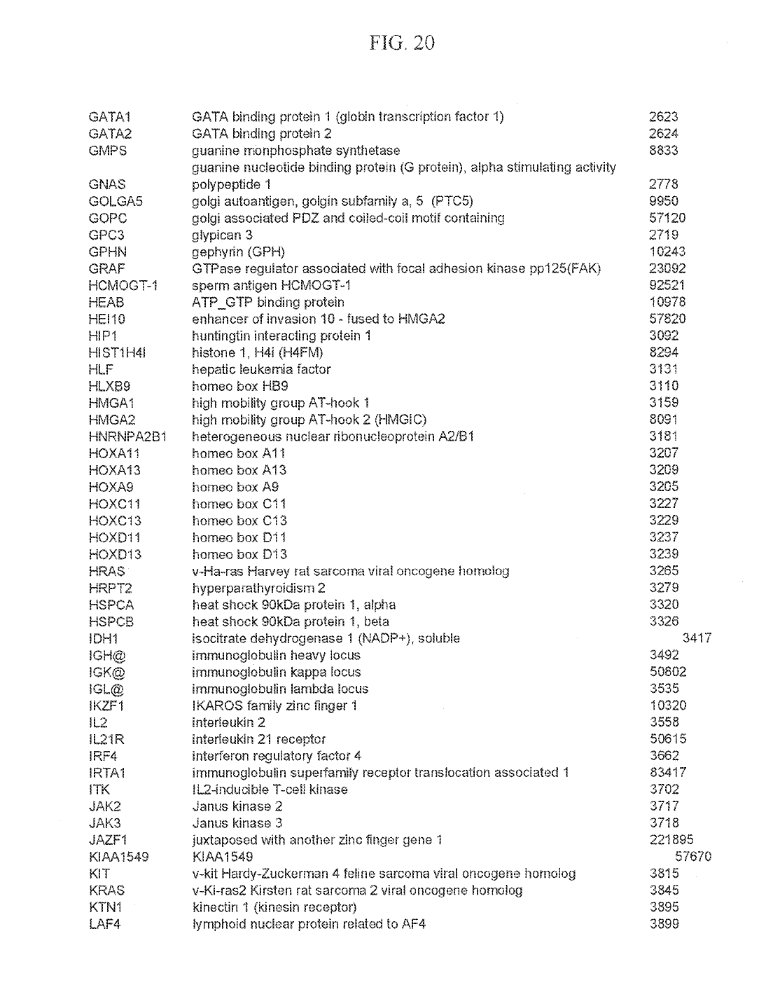

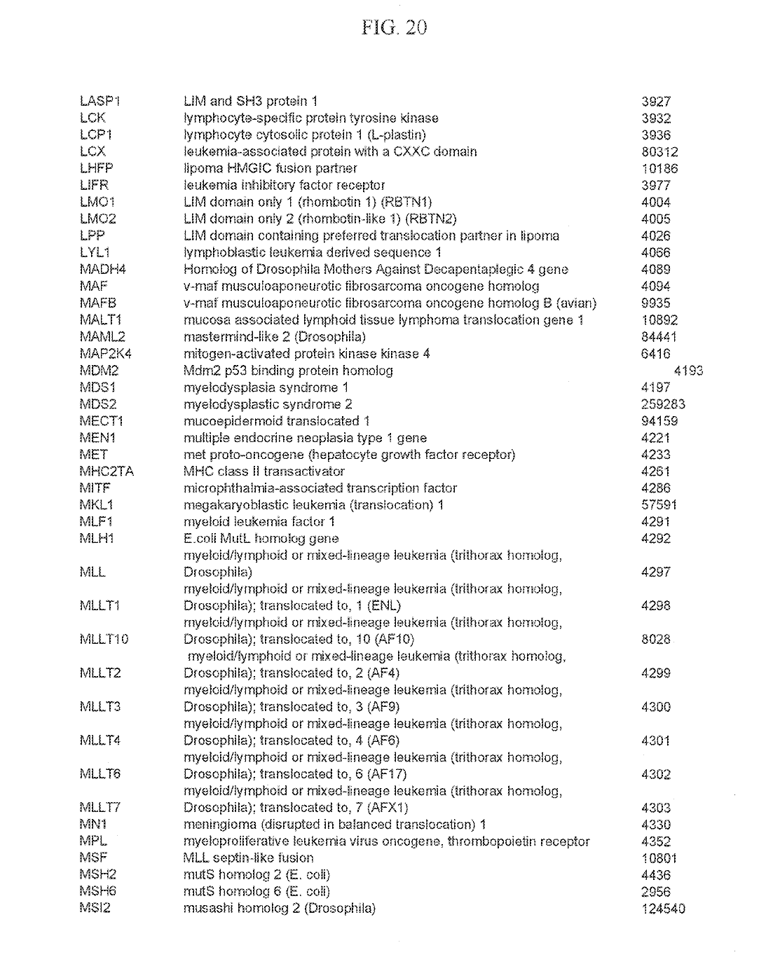

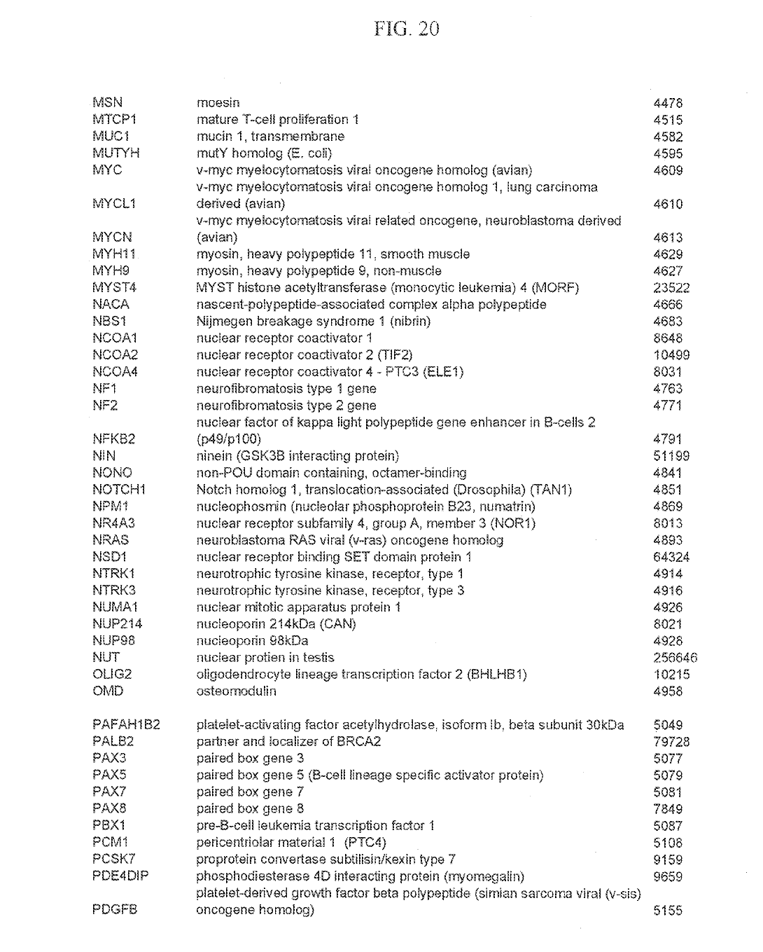

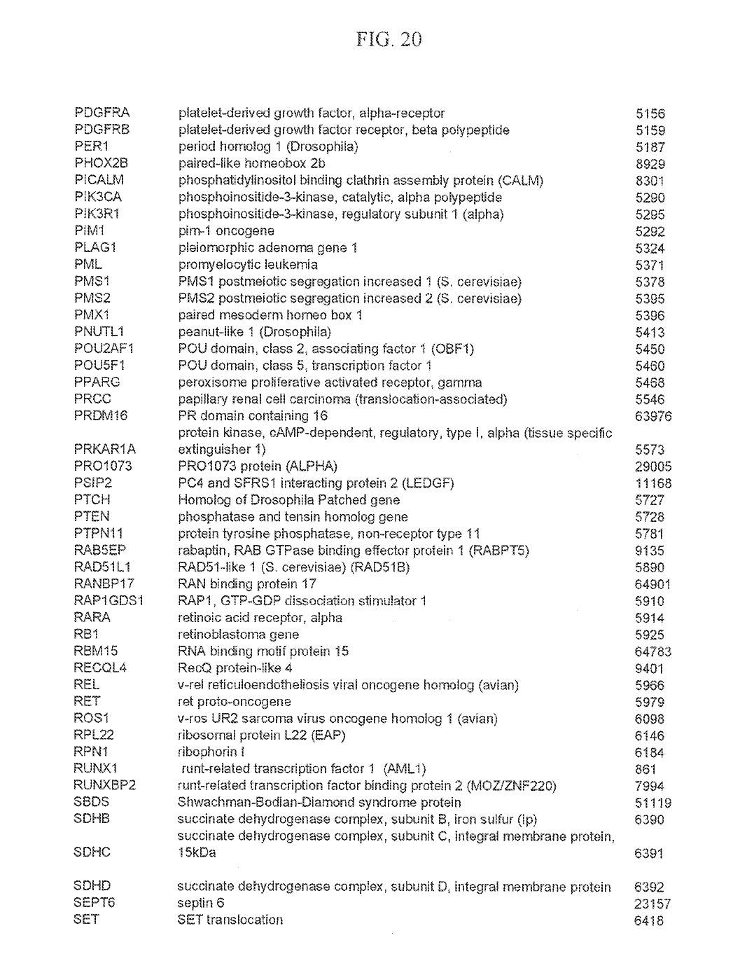

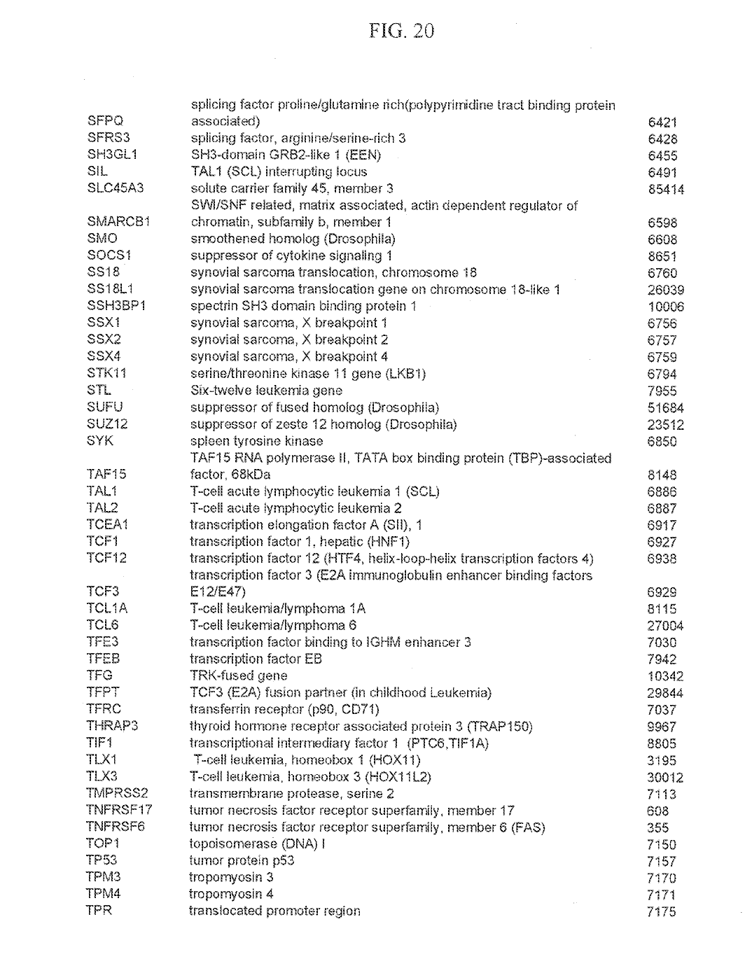

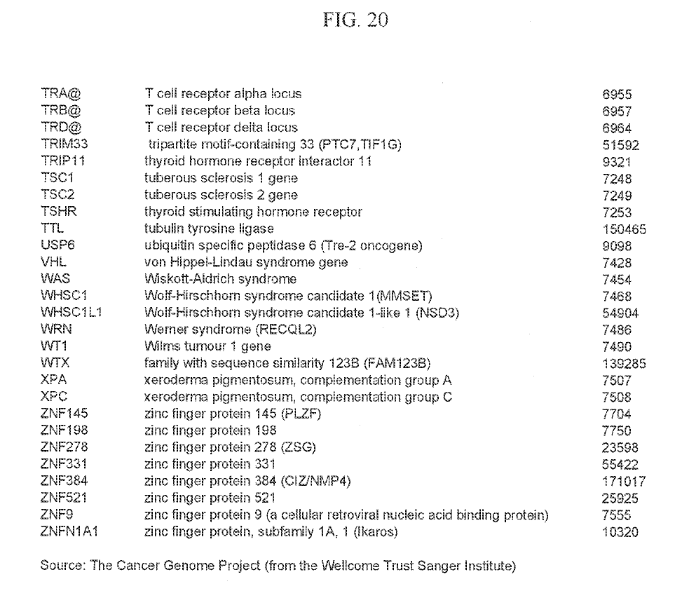

Detection of gene expression: The present invention provides implantable compositions, such as cell engrafted biocompatible matrices, which can modulate cancer cell virulence and tumor growth. The effectiveness of the composition of the invention can be determined by assaying the expression level of cancer cell biomarkers—i.e., target genes which are indicative of cancer cell phenotypes, such as proliferation, virulence, metastasis, and invasiveness. Changes in gene expression (e.g., gene expression profiling) can be linked to specific effects (or classes/types of effects) on cells and therefore can be used to modify or customize cancer treatment. For example, downregulation of Twist or Snail or Slug can be indicative of decreased invasiveness, upregulation of p53 (if functional) can be indicative of increased cancer cell apoptotic death or cell cycle arrest. In response, a patient's treatment can be modified to maximize therapeutic benefit. Biomarkers linked to cancer cell phenotypes include, for example, genes involved in the epithelial-mesenchymal transition (e.g., E-cadherin, N-cadherin, Vimentin, Snail, Slug) and genes associated with stem-cell phenotypes (e.g., CD133, ABCG2). Other biomarkers include, for example, STAT1, STAT2, STAT3, STAT4, STAT5, STAT6, JAK1, JAK2, Twist, Snail, Slug, Sipl, Ki67, PCNA, N-cadherin, fibronectin, VEGF, FGF, HGF, EGF, IGF, TGF-beta, BMP, versican, and perlecan. A non-limiting list of other possible markers of cancer cellular virulence is provided in FIG. 20 (Wellcome Trust, London). Likewise, gene expression of cells engrafted in biocompatible matrices can be monitored for expression of factors that modulate cancer cell phenotypes. Many methods of detection of a protein, nucleic acid, or activity level of interest, with or without quantitation, are well known and can be used in the practice of the invention.

Target gene transcripts can be detected using numerous techniques that are well known in the art. Some useful nucleic acid detection systems involve preparing a purified nucleic acid fraction of a sample (e.g., a tumor biopsy, a cancer cell culture, a cell engrafted biocompatible matrix) and subjecting the sample to a direct detection assay or an amplification process followed by a detection assay. Amplification can be achieved, for example, by polymerase chain reaction (PCR), reverse transcriptase (RT), and coupled RT-PCR. Detection of a nucleic acid can be accomplished, for example, by probing the purified nucleic acid fraction with a probe that hybridizes to the nucleic acid of interest, and in many instances detection involves an amplification as well. Northern blots, dot blots, microarrays, quantitative PCR, quantitative RT-PCR, and real-time PCR are all well known methods for detecting a nucleic acid in a sample. Nucleic acids also can be amplified by ligase chain reaction, strand displacement amplification, self-sustained sequence replication or nucleic acid sequence-based amplification. Nucleic acids can also be detected by sequencing; the sequencing can use a primer specific to the target nucleic acid or a primer to an adaptor sequence attached to the target nucleic acid. Sequencing of randomly selected mRNA or cDNA sequences can provide an indication of the relative expression of a biomarker as indicated by the percentage of all sequenced transcripts containing nucleic acid sequence corresponding to the biomarker. Alternatively, a nucleic acid can be detected in situ, such as by hybridization, without extraction or purification. Gene transcripts can be detected on a medium-throughput basis, such as by using a qRT-PCR array (e.g., RT2 Endothelial Cell Biology PCR Array; SABiosciences, Baltimore Md.). In addition, target gene transcripts can be detected on a high-thoughput basis using a number of well known methods, such as cDNA microarrays (Affymetrix, Santa Clara, Calif.), SAGE (Invitrogen, Carlsbad, Calif.), and high-throughput mRNA sequencing (Illumina Inc., San Diego, Calif.).

Target proteins can be detected, for example, immunologically using one or more antibodies. In immunological assays, an antibody having specific binding affinity for a biomarker or a secondary antibody that binds to such an antibody can be labeled, either directly or indirectly. The antibody need not be complete: an antibody variable domain or an artificial analog thereof, such as a single chain antibody, is sufficient. Suitable labels include, without limitation, radionuclides (e.g., ¹²⁵I, ¹³¹I, ³⁵S, ³H, ³²P, or ¹⁴C), fluorescent moieties (e.g., fluorescein, FITC, perCP, rhodamine, or PE), luminescent moieties (e.g., Qdol™ nanoparticles supplied by the Quantum Dot Corporation, Palo Alto, Calif.), compounds that absorb light of a defined wavelength, or enzymes (e.g., alkaline phosphatase or horseradish peroxidase). Antibodies can be indirectly labeled by conjugation with biotin then detected with avidin or streptavidin labeled with a molecule described above. Methods of detecting or quantifying a label depend on the nature of the label and are known in the art. Examples of detectors include, without limitation, x-ray film, radioactivity counters, scintilation counters, spectrophotometers, colorimeters, fluorometers, luminometers, and densitometers. Combinations of these approaches (including “multi-layer” assays) familiar to those in the art can be used to enhance the sensitivity of assays.

Immunological assays for detecting a target protein can be performed in a variety of known formats, including sandwich assays, competition assays (competitive RIA), or bridge immunoassays. See, for example, U.S. Pat. Nos. 5,296,347; 4,233,402; 4,098,876; and 4,034,074. Methods of detecting a target protein generally include contacting a biological sample with an antibody that binds to the protein and detecting binding of the protein to the antibody. For example, an antibody having specific binding affinity for a target protein can be immobilized on a solid substrate by any of a variety of methods known in the art and then exposed to the biological sample. Binding of the target protein to the antibody on the solid substrate can be detected by exploiting the phenomenon of surface plasmon resonance, which results in a change in the intensity of surface plasmon resonance upon binding that can be detected qualitatively or quantitatively by an appropriate instrument, e.g., a Biacore® apparatus (Biacore International AB, Rapsgatan, Sweden). Alternatively, the antibody can be labeled and detected as described above. A standard curve using known quantities of a protein can be generated to aid in the quantitation of biomarker levels.

In other embodiments, a “sandwich” assay in which a capture antibody is immobilized on a solid substrate is used to detect the level of a target protein. The solid substrate can be contacted with the biological sample such that any target protein in the sample can bind to the immobilized antibody. The level of the target protein bound to the antibody can be determine using a “detection” antibody having specific binding affinity for the target protein and the methods described above. It is understood that in these sandwich assays, the capture antibody should not bind to the same epitope (or range of epitopes in the case of a polyclonal antibody) as the detection antibody. Thus, if a monoclonal antibody is used as a capture antibody, the detection antibody can be another monoclonal antibody that binds to an epitope that is either completely physically separated from or only partially overlaps with the epitope to which the capture monoclonal antibody binds, or a polyclonal antibody that binds to epitopes other than or in addition to that to which the capture monoclonal antibody binds. If a polyclonal antibody is used as a capture antibody, the detection antibody can be either a monoclonal antibody that binds to an epitope that is either completely physically separated from or partially overlaps with any of the epitopes to which the capture polyclonal antibody binds, or a polyclonal antibody that binds to epitopes other than or in addition to that to which the capture polyclonal antibody binds. Sandwich assays can be performed as sandwich ELISA assays, sandwich Western blotting assays, or sandwich immunomagnetic detection assays.

Suitable solid substrates to which an antibody (e.g., a capture antibody) can be bound include, without limitation, microtiter plates, tubes, membranes such as nylon or nitrocellulose membranes, and beads or particles (e.g., agarose, cellulose, glass, polystyrene, polyacrylamide, magnetic, or magnetizable beads or particles). Magnetic or magnetizable particles can be particularly useful when an automated immunoassay system is used.

Other techniques for detecting target polypeptides include mass-spectrophotometric techniques such as electrospray ionization (ESI), and matrix-assisted laser desorption-ionization (MALDI). See, for example, Gevaert et. al. (2001) Electrophoresis 22(9):1645-51; Chaurand et al. (1999) J. Am. Soc. Mass Spectrom 10(2):91-103. Mass spectrometers useful for such applications are available from Sigma (St. Louis, Mo.); Applied Biosystems (Foster City, Calif.); Bruker Daltronics (Billerica, Mass.); and GE Healthcare (Piscataway, N.J.). In addition, target proteins can be detected on a high-thoughput basis using protein microarrays (Invitrogen; Carlsbad, Calif.).

It will be appreciated that the expression of any target gene transcript or target protein according to the present invention can be readily detected using one or more of the above techniques.

The following Methods, Materials, and Examples are provided for illustration, not limitation.

### Experimental Materials and Methods

### 1: Endothelial Cell Culture

Primary human aortic endothelial cells (HAEC), human umbilical vein endothelial cells (HUVEC), and human dermal microvascular endothelial cells (HMVEC-d) are purchased from Lonza (Basel, Switzerland), Invitrogen (Eugene, Oreg.) or Cascade Biologics (Portland, Oreg.) and used between passages 3 and 9 (or 2 through 7 for HUVEC). The culture medium (“endothelial cell growth medium”) for all adult endothelial cell types is a 1:1 mixture of EGM2 (Lonza, Switzerland; containing EGF, hydrocortisone, gentamicin, amphotericin-B, FBS to 5% final volume, VEGF, FGF02, IGF-1, ascorbic acid, and heparin) with an extra 3% FBS and EGM2-MV. Human adult peripheral blood endothelial progenitor cells are isolated from late outgrowth colonies from the mononuclear cell (MNC) fraction of blood as described in Broxmeyer et al., “Cord blood stem and progenitor cells,” Methods Enzymol, 419;439-73 (2006). Briefly, 5×10⁷ blood MNC are plated per well of 6-well collagen I-coated tissue culture plates with EGM2 media with a total of 20% FBS. After 24 hours, nonadherent cells are gently rinsed off and fresh media is added. Media is changed every 24 hours for the first 7 days, and every 48 hours thereafter. Endothelial progenitor cells are harvested from endothelial colonies appearing between days 7 and 21 in culture.

All endothelial cells are cultured on gelatin-coated tissue culture polystyrene (TCPS) plates in a 37° C., humidified, 54% CO₂ environment; medium is changed every 48-72 hours. Gelatin is purchased as a 0.1% solution (Millipore). Cells are passaged by trypsinization and splitting about 1 to 6. For endothelial conditioned media collection, the culture medium is either endothelial cell growth medium or EBM2 (Lonza, Switzerland) supplemented with 0.5% FBS, 100 U/mL penicilin, and 100 μg/mL streptomycin.

In one embodiment, cell engrafted biocompatible matrices are prepared by culturing cells on Gelfoam® compressed sponge (Pfizer, New York, N.Y.). After cutting the Gelfoam® into 2.5×1×0.3 cm blocks, Gelfoam® blocks are hydrated in endothelial cell growth medium at 37° C. for about ≧4 hours (but fewer than 48 hours). 9×10⁴ endothelial cells (suspended in about 100 μL endothelial cell growth medium) are seeded onto hydrated Gelfoam® blocks and allowed 3 hours to attach before adding each piece to a separate 30 mL polypropylene tube containing 6 mL of endothelial cell growth medium. Cell engrafted biocompatible matrices are cultured for up to 3 weeks, with media changed every 48-72 hours, under standard culture conditions (37° C. humidified environment with 5% CO₂). Engrafted cells are released from the Gelfoam® matrix by digestion with 1-2 mg/mL collagenase type I or type IV (Worthington Biochemicals, Freehold, N.J.) following 2 washes in PBS to remove nonadherent cells and serum.

Examples of preferred configurations suitable for use in this manner are disclosed in U.S. patent application Ser. No. 11/792,350, based on International Patent Application No. PCT/US05/43967, filed on Dec. 6, 2005, the entire contents of each of which are herein incorporated by reference. Related flowable compositions suitable for use in accordance with the present invention are disclosed in U.S. patent application Ser. No. 11/792,284, based on International Patent Application No. PCT/US05/43844, filed on Dec. 6. 2005, the entire contents of each of which are herein incorporated by reference.

### 2: Cancer Cell Culture

All human cancer lines are purchased from the American Type Culture Collection (ATCC) unless otherwise noted. Cancer cells are cultured in either DMEN (SK-LMS-1, SK-UT-1, AS49) or RPM11640 (NC1-H520) supplemented with 100 U/mL penicilin, 100 μg/mL streptomycin, and 10% v/v FBS. All human cancer cells are cultured on TCPS plates or flasks in a 37° C. humidified environment with 5% CO₂. Cells are passaged by trypsinization (or 5 mM EDTA treatment) and splitting about 1 to 8.

### 3: Characterization of Cultures

Optical Microscopy

Optical imaging of cell cultures, to track gross morphology and health, will be performed with a Nikon phase contrast microscope (with attached Nikon digital camera). A Leica microscope with attached computer/camera interface will be used to record the motile behavior of cells (i.e., for in vitro chemoinvasion assays. Images will be analyzed with Photoshop® CS3 (Adobe; San Jose, Calif.) and ImageJ (National Institutes of Health).

Confocal Laser Scanning Microscopy

Expression of endothelial and cancer cell surface markers will be analyzed by confocal microscopy. Cells are seeded on coverslips or embedded in Gelfoam® matrices. After washing with PBS and fixation with 4% paraformaldehyde for 20 minutes (cover slips) or overnight (Gelfoam® matrices), cells are blocked with rat serum (Bethyl Laboratories) for 30 minutes. Before staining with antibodies, Gelfoam® matrices are cut into 2-mm thick slices. Endothelial cells are strained with the appropriate amount of antibodies for 1 (cover slips) or 2 hours (Gelfoam® matrices) and analyzed on a Zeiss LSM510 Laser scanning confocal microscope. Staining intensity is quantified with ImageJ (National Institutes of Health) and normalized against CD31 (endothelial cells) or other housekeeping gene (cancer cells) expression.

Cell Number

The concentration of cell suspensions (harvested by trypsinizing or by incubation with EDTA) is measured by a Z1 Coulter particle counter (Beckman Coulter; Fullerton, Calif.). Cells can also be counted manually using a hemacytometer.

Cell Viability

Cell viability is determined by trypan blue exclusion—followed by counting the fraction of dead cells, which take up the dye, using a hemacytometer—or via a Live/Dead viability/cytotoxicity kit (Invitrogen; Carlsbad, Calif.), in which membrane-permeant calcein is cleaved by cytosolic enzymes to yield a green fluorescent signal in live cells or membrane-impermeant ethidium homodimer binds to nucleic acids of dead cells to yield a red fluoresent signal.

Cell Proliferation

Proliferation is measured using an MTS-based assay (CellTiter Aqueous One Proliferation Assay; Promega, Madison, Wis.). Cells are cultured in 96-well optical plates in 100 μL of appropriate medium. 20 μL of MTS reagent is added to each well and the plate is incubated at 37° C. for 1 hour, after which the absorbance at 490 nm is measured with a Ceres UV900 HDi multichannel spectrophotometer (BioTek Instruments, Winooski, Vt.). All experimental conditions will be tested, at the minimum, in triplicate.

Alternatively, proliferation will be measured using ³H-thymidine incorporation. Cell cultures are incubated under standard conditions (37° C., 5% CO₂) and pulsed with ³H-thymidine (1 μCi/mL, 2 hours, Perkin Elmer Life Sciences). Cultures are washed twice with 2 mL of ice cold PBS followed by 30 minutes incubation in 5% wt/vol trichloroacetic acid (TCA). TCA is washed twice with cold PBS, followed by lysis with 0.4 mL of lysis solution (0.5% SDS, 0.5 N NaOH). The TCA-insoluble radioactivity is measured in a liquid scintilation counter (Packard 25000-TR).

Apoptosis

Apoptosis is quantified with a caspase fluorimetric assay (Apo-ONE caspase-3/7 assay, Promega, Madison, Wis.). Cells are cultured in 96-well optical plates (coated with type I collagen if culturing cancer cells) in 100 μL of appropriate medium. Caspase detection reagent will be prepared and added to the cultured cells as recommended by the manufacturer. After 1-2 hours incubation with the reagent, the fluorescence (499 nm excitation, 521 nm emission) is measured using a multichannel fluorimeter (Fluoroscan Ascent FL, Thermo Fisher Scientific, Waltham, Mass.) Alternatively apoptosis will be detected by Annexin V/PI or TUNEL staining and flow cytometric analysis.

Cell Cycle

Cell cultures are pulsed with BrdU (10 μM, 6 hours; Pharmingen, San Diego, Calif.), then washed 3 times in ice cold PBS followed by 20 minutes incubation in 1 mL of Carnoy fixative (4° C.) and acid DNA denaturation (HCI 2 M, 37° C., 1 hour). BrdU is then labeled by immunostaining using Alexa Fluor® 594 conjugate anti-BrdU antibody. The amount of BrdU incorporated is then compared with the total DNA content measured by propidium iodide (PI; Molecular Probes, Eugene, Oreg.).

RNA

Total RNA is extracted from cells using the RNEasy® Mini Kit (Qiagen, Valencia, Calif.). Complementary DNA is synthesized using TaqMan® reverse transcription reagents (Applied Biosystems; Foster City, Calif.). Real-time PCR analysis is performed with an Opticon™ Real Time PCR Machine (MJ Research, Waltham, Mass.) using SYBR® Green PCR Master Mix (Applied Biosystems, Foster City, Calif.) and appropriate primers. Reaction data are collected and analyzed by the complementary Opticon™ computer softward. Relative quantification of gene expression is calculated with standard curves and normalized to GAPDH.

Protein

Whole cell extracts are harvested by repeated washes with EDTA (2.5 mM in PBS, 2 minutes). Protein samples are separated on glycine-SDS gels, transferred to polyvinylidene fluoride (PVDF) membranes, and immunobioted with the appropriate chemiluminescent antibodies as recommended by the manufacturer. Gel luminescence is measured by a FluorChem® luminometer (Alpha Innotech; San Leandro, Calif.).

Expression levels of cell surface markers of cultured cells are quantified by flow cytometry. Cultures are harvested (PBS, EDTA 5 mM, 15 minutes) and labeled with fluorescein isothiocyanate conjugated (FITC), Phycoerythrin (PE), or other fluorochorome-labeled antibodies. Labeled cells are analyzed with a FACScan™ flow cytometer (Becton Dickinson, Franklin Lakes, N.J.), using at least 10,000 positive events. Listmode files are analyzed using FlowJo software (TreeStar, Ashland, Oreg.).

### 4: Quantitative Assessment of Cell Biosecretions

Total protein production is determined by a bioinchoninic acid (BCA) protein assay kit (Pierce, Rockford, Ill.). Total glycosaminoglycan and heparan sulfate proteoglycan production are determined using a dimethylmethylene blue assay before and after cell-conditioned medium treatment with chondroitinase ABC (0.1 U/sample, Seikagaku America) for 3 hours at 37° C. to eliminate chondroitin and dermatan sulfate. Prostacyclin concentrations are determined by a 6-ketoprostaglandin F1 ELISA assay (Assay Designs, Ann Arbor, Mich.). Transforming growth factor-β (TGF-β) and endothelin are measured using standard ELISA assays (Assay Designs, Ann Arbor, Mich.). All assay kits are used according to manufacturers' instructions.

### 5: Protein Expression and Western Blotting

Whole cell extracts were harvested with lysis buffer containing 0.5% Triton X-100, 0.1% SDS, and inhibitors of proteases and phosphatases (Roche protease inhibitor tablet, 2 mM sodium orthovanadate, 50 mM sodium fluoride, 4 mM PMSF). Protein samples were separated on glycine-SDS gels, transferred to nitrocellulose membranes, immunoblotted with the appropriate primary antibodies, followed by HRP-conjugated secondary antibodies and a chemiluminescent detection reagent (SuperSignal Femto, Pierce). Gel luminescence was measured by a FluorChem luminometer (Alpha Innotech; CA) and analyzed using ImageJ.

A cytokine antibody array (RayBiotech; GA) was used following the manufacturer's instructions for assessment of cell biosecretions. Array luminescence was imaged using a FluorChem luminometer (Alpha Innotech; CA) and analyzed using ImageJ.

### 6. Reagents

Primary antibodies targeting Ki67, MMP2, and -actin were purchased from Santa Cruz Biotechnology, primary antibodies targeting NF-kB p65, p-S6RP and p-STAT3 were purchased from Cell Signaling Technology, and the primary antibody targeting PCNA was purchased from Abcam HRP-conjugated secondary antibodies were purchased from Santa Cruz Biotechnology. Fluorescently-labeled secondary antibodies were purchased from Invitrogen. Rapamycin was purchased from Sigma Oligonucleotide PCR primers were purchased from Invitrogen. DAPI was purchased from Invitrogen.

### 7. Immunofluorescent Staining and Epifluorescence Microscopy

Cells were washed, fixed (10 minutes, 4% paraformalehyde, room temperature), permeabilized with 0.25% Triton X-100, and incubated with primary antibodies overnight at 4° C. in a humidified chamber. Fluorescently-labelled secondary antibodies were then added, along with 2.5 mg/mL DAPI, for two hours at room temperature in the dark. Cells were then washed and coverslipped with antifade mounting media (ProLong Gold, Invitrogen). Stained cells were imaged using an epifluorescence microscope (Leica) and analyzed using ImageJ.

Excised rumors were flash frozen in liquid N₂ cooled isopentane. 10-μm frozen sections were cut using a cryotome, fixed for 10 minutes with acetone at −20° C., blocked with serum/BSA/PBS for 45 minutes at room temperature, and stained with appropriate primary and fluorescence-conjugated antibodies as described for cells.

### 8. Statistical Analysis

All assays are repeated three times and experiments are run at least three times; results are expressed as mean +/− standard deviation. Comparison of two groups is performed using a student's test (appropriately assuming identical or differing variance, depending on the circumstance). Comparison of multiple (≧2) groups (with the same assumed variance) is performed by using analysis of variance (ANOVA) followed by a student's t-test or the Bonferroni correction when necessary. Statistical software used in these analyses include Excel® (Microsoft, Redmond, Wash.), MATLAB® (Mathworks, Natick, Mass.), and JMP® (SAS, Cary, N.C.). P<0.05 is taken as statistically significant.

### EXAMPLES

### Example 1

### Endothelial Cell Conditioned Media Modulates Cancer Cell Proliferation

The effects of EC-conditioned media on cancer cell proliferation were examined during exponential growth in culture. Primary human umbilical vein endothelial cells (HUVECs, Invitrogen) were cultured on gelatin-coated TCPS plates and used between passages 2-6. The culture medium (“EC growth medium”) for HUVECs was EGM2 (Lonza) with an additional 3% FBS. Cells were passaged by detachment with trypsin and split 1 to about 5. Endothelial cell conditioned media was generated by 48 hours of culture in MDCB (Invitrogen) supplemented with 10% FBS, 100 U/mL penicilin, and 100 μg/mL streptomycin. Cells and debris were removed by centrifugation (5 minutes, 500 g) and endothelial cell conditioned media were aliquotted and stores at −80° C. A A549 (large cell lung carcinoma cells) and MDA-MB-231 (breast carcinoma cells) were purchased from ATCC. Cancer cells were cultured on TCPS dishes in a 37° C., humidified, 5% CO2 environment; medium was changed every 48-72 hours.

After 96 hours of culture, with a media change after 48 hours, adherent cells were detached and then counted with a particle counter.

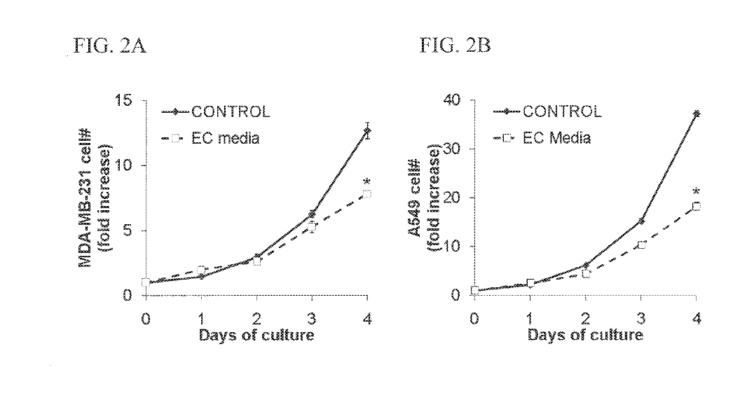

Proliferation curves for cancer cell lines cultured in endothelial cell media or control media are shown in FIG. 2A (MDA-MB-231 cells) and FIG. 2B (A549 cells). As shown in FIGS. 2A and B, EC-conditioned media reduced cancer cell proliferation.

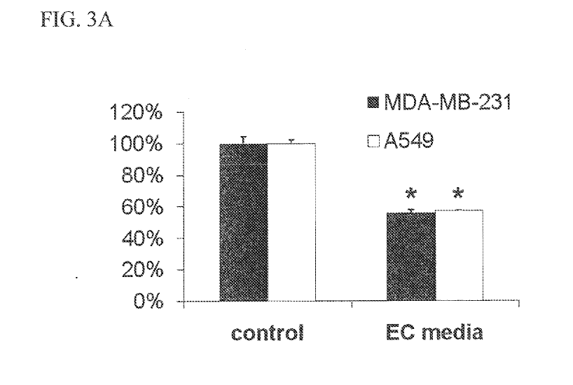

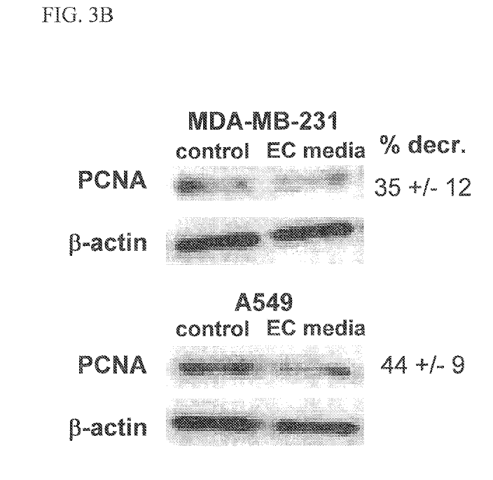

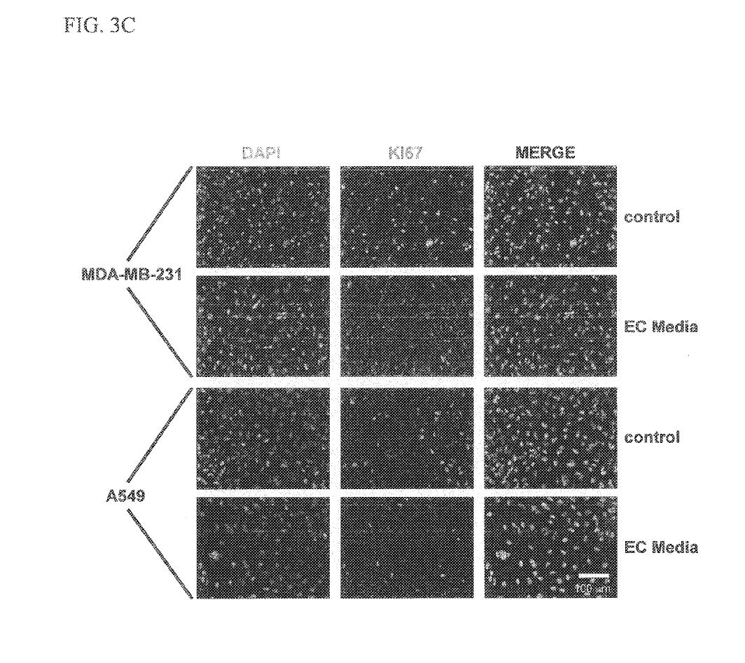

Media conditioned by healthy confluent endothelial cells reduced significantly the proliferation of both breast carcinoma (MDA-MB-231) cells and lung carcinoma cells (A549; FIG. 3A). The reduction of cancer cell proliferation by culture in endothelial cell secretions was consistent with decreased expression of cancer cell PCNA by Western blot (FIG. 3B) and with decreased fraction of cancer cells with Ki-67 positive nuclei (FIG. 3C).

As shown in FIG. 3A, MDA-MB-231 and A549 proliferation is reduced by about 45% after culture for 96 hours in endothelial cell conditioned media. As shown in FIG. 3B, expression of PCNA in cancer cells decreases by about 40% after 96 hours of culture in endothelial cell conditioned media. As shown in FIG. 3C, expression of Ki67 in cancer cells decreases by about 30% after 96 hours of culture in endothelial cell conditioned media. The expression of PCNA (FIG. 3B) and Ki-67 (FIG. 3C) proteins, two markers of cellular proliferation, correlated with effects on proliferation.

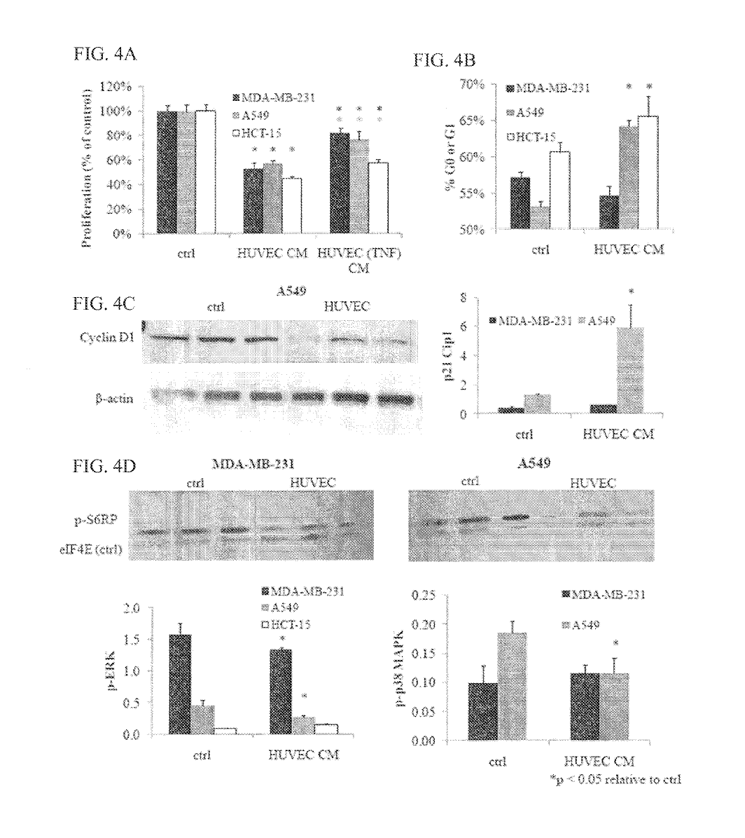

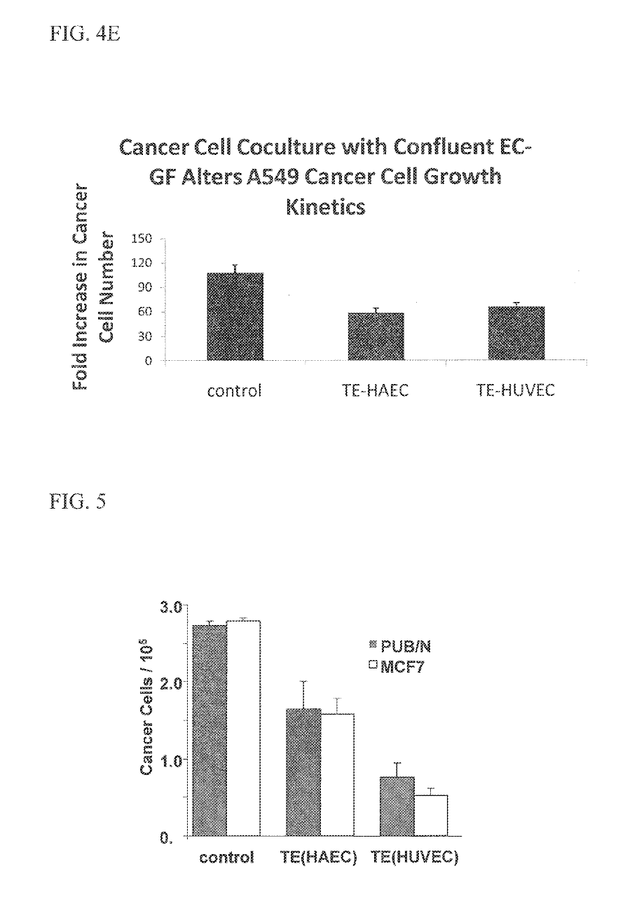

As shown in FIG. 4A, cancer cell proliferation is significantly attenuated when cancer cells are cultured in media conditioned by healthy endothelial cell, but less so for endothelial cell pretreated with 10 ng/mL of TNF-α, for 96 hours.

As shown in FIG. 4B, cell cycle progression is significantly attenuated when cancer cells are cultured in healthy endothelial cell conditioned media for 96 hours.

As shown in FIG. 4C, cell cycle proteins show characteristics of cell cycle arrest when cancer cells are cultured in healthy endothelial cell conditioned media for 72-96 hours.

As shown in FIG. 4D, proliferation associated signaling proteins are less stimulated after culture with healthy endothelial cell conditioned media for 96 hours.

To demonstrate that engrafted endothelial cells also can modulate cancer cell proliferation in vitro, HAEC and HUVEC were engrafted on Gelfoam® as described herein and were then co-cultured with A549 cells. As shown in FIG. 4E, co-culture of A549 cells with engrafted (TE) HAEC and HUVEC reduces cancer cell proliferation.

These data suggest that healthy endothelial cells secrete factors that suppress cancer cell proliferation.

### Example 2

### Engrafted Endothelial Cell Conditioned Media Modulates Cancer Cell Proliferation

To confirm these modulatory effects, cancer cell proliferation and invasiveness were further assessed in vitro in response to media conditioned with engrafted endothelial cells and media conditioned with “late-outgrowth” endothelial progenitor cells (EPCs) to demonstrate that engrafted endothelial cells can inhibit cancer cell proliferation and virulence. Briefly, cancer cell proliferation (tumor growth) was analyzed via MTS assay, and cancer cell invasiveness (metastasis) was analyzed via chemoinvasiion assay. Two well-differentiated cancer lines, SK-LMS-1 leiomyosarcoma and NCI-H520 squamous lung carcinoma were used. Endothelial cells in various states (e.g., subconfluent, post confluent) and from various vascular beds were used.

SK-LMS-1 and NCI-H520 cancer cells were cultured as described above. Functional assays as described above were used to analyze the cancer cell phenotype before and after culture with media conditioned from a selected group of endothelial cells. This selected group included HAEC and HUVEC (large vessel endothelial cells, which have regulatory properties in vascular regeneration and which show differential secretion of key regulatory molecules), HMVEC-d (dermal microvascular endothelial cells), and, in certain studies, adult peripheral blood endothelial cell progenitors (circulating cells that are recruited from the bone marrow and incorporated into nascent vasculature (see Hirschi, “Arterioscler, Thromb, Vasco, Biol., 28(9):1584-95 (2008) as these cell types exhibit variable control over vascular repair. In certain studies, the endothelial progenitor cells were classified as “late-outgrowth” cells to distinguish them from “early-outgrowth” progenitor cells that are more monocyte-like.

Matrix engrafted endothelial cells as described above have a significant regulatory role on cancer cell proliferation. As illustrated in FIG. 5, matrix engrafted endothelial cells (TE) inhibited proliferation of co-cultured PUB/N lung carcinoma and MDF7 breast adenocarcinoma cell lines in vitro. The anti-proliferative effects of matrix engrafted endothelial cells were dependent on the vascular bed of origin of the endothelial cells (HUVECs had more of an anti-proliferative effect than HAECs).

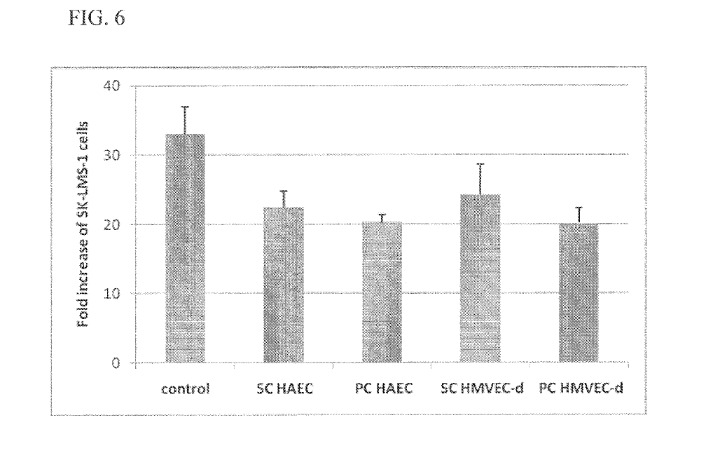

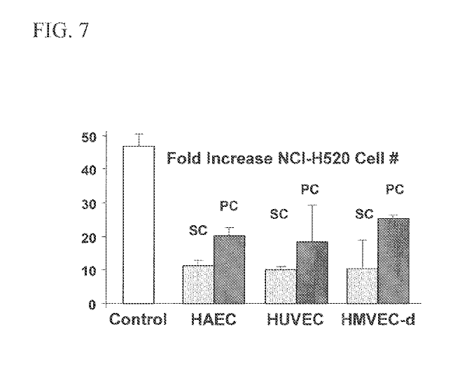

As illustrated in FIG. 6, media conditioned with matrix-engrafted endothelial cells as described above inhibited proliferation of cancer cells in a cell density- and vascular bed origin-dependent manner. Specifically, the proliferation of SK-LMS-1 leiomyosarcoma cells, assayed via the above-described MTS assay, was significantly decreased relative to control (empty Gelfoam® matrices) after 6 days of culture in the presence of media conditioned from engrafted human aortic endothelial cells (HAEC) regardless of cell density (SC=subconfluent versus PC=postconfluent), whereas media conditioned from HMVEC-d (dermal microvascular endothelial cells) showed density-dependent control of SK-LMS-1 proliferation. Thus, engrafted endothelial cells are capable of inhibiting cancer cell proliferation.

As illustrated in FIG. 7, the proliferation of NCI-H520 squamous lung carcinoma cells, as measured using an MTS assay, was suppressed after 6 days in culture in the presence of media conditioned by HAEC, HUVEC, and HMVEC-d cells. Proliferation of NCI-H520 was suppressed the most by subconfluent (SC) endothelial cells, but also suppressed by postconfluent (PC) endothelial cells.

It is believed that the proliferation of cancer cells cultured in conditioned media from healthy endothelial cells will be attenuated by induction of cell cycle arrest rather than apoptosis. The following assays will be used: Live/Dead stain and trypan blue exclusion for estimation of cell viability, MTS assay or 3H-thymidine incorporation for cancer cell proliferation, BrdU/PI flow cytometry for cell cycle analysis, fluorimetric caspase assay or Annexin V/PI flow cytometry for apoptosis quantification. Moreover, a chemoinvasion assay (BD Biocoat Matrigel Invasion chamber; Becton Dickinson, Franklin Lake, N.J.), e.g., as described in Albini, “The chemoinvasion assay: a method to assess tumor and endothelial cell invasion and its modulation.” Nat. Protoc., 2(3):504-11 (2007), will be used to study the invasiveness of cancer cells before and after culture with endothelial cell conditioned media.

### Example 3

### Plated and Engrafted Endothelial Cells Regulate Cancer Cell Invasiveness

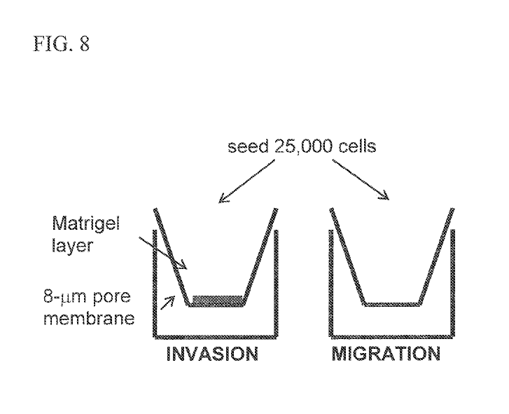

Cancer cell invasiveness is a key trait in determining the aggressiveness and metastatic potential of tumors. Thus, this property was examined using a chemoinvasion/chemomigration assay, to analyze how cancer cells chemotax through cell culture insert pores which had been either coated with extracellular matrix proteins (to emulate “invasion”) or uncoated (to emulate “migration”). FIG. 8, shows a schematic diagram of a chemoinvasion/chrmomigration assay. Proliferation was measured by harvesting adherent cells and counting the cell suspension concentration with a Coulter counter (Beckman Coulter, Fullerton, Calif.) Briefly, commercially available chemoinvasion chamber kits (BioCoat, Becton Dickinson) were used according to the manufacturer's instructions. Invades or migrated cells adherent to the bottom of the assay's inserts are fixed, stained with DAPI and imaged with an epifluorescence microscope. The invasion index is calculated as the average number of invaded cells divided by the average number of migrated cells of a given condition.

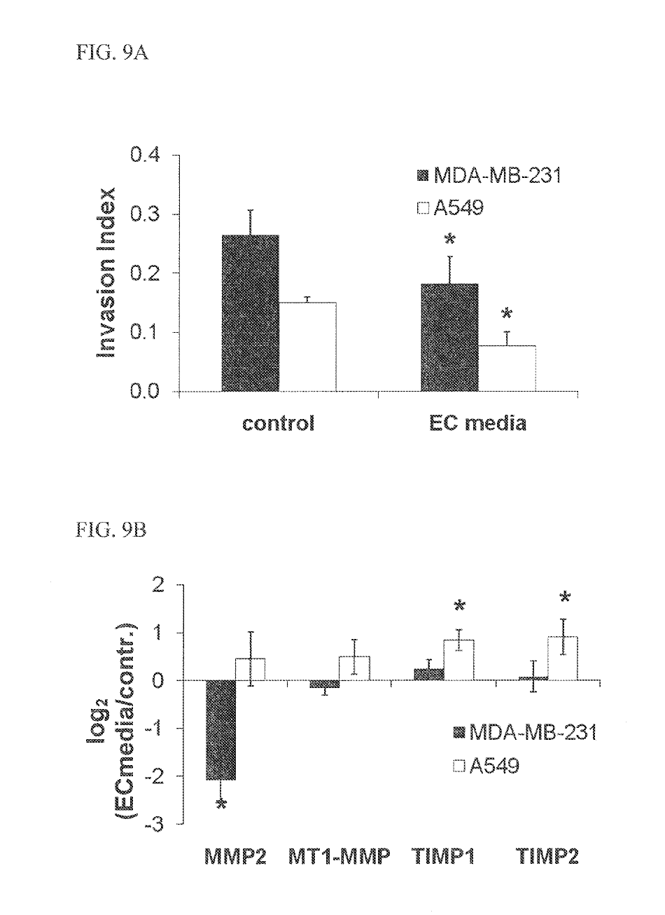

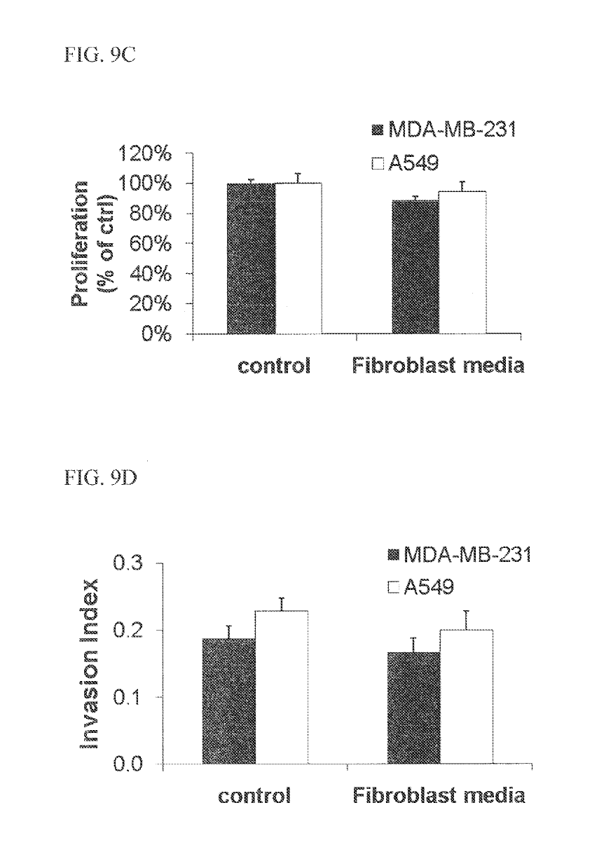

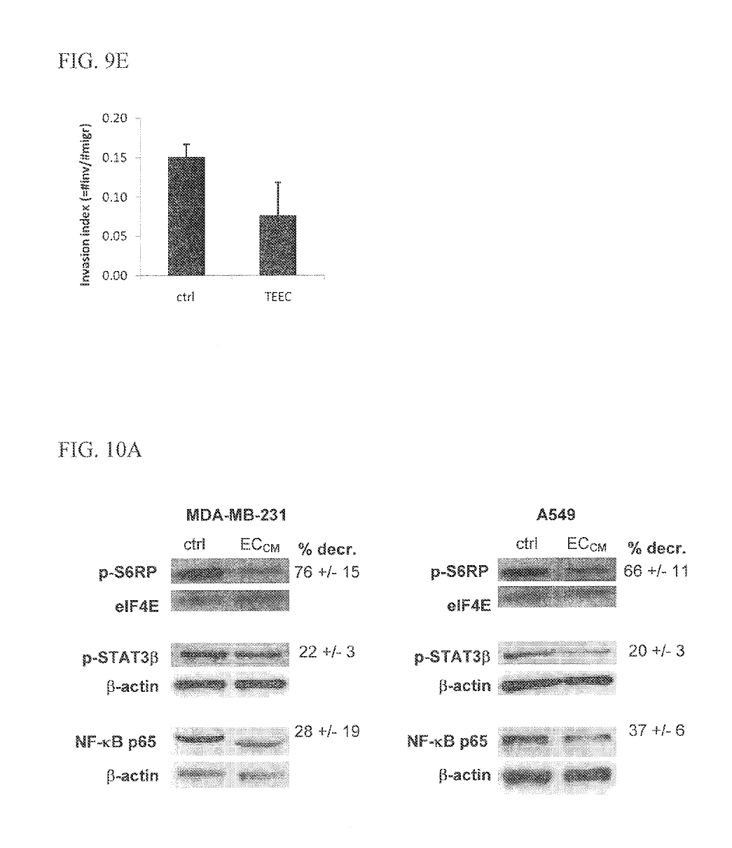

As shown in FIG. 9A, MDA-MB-231 cells and A549 cells are about 40% less invasive than control cells after culture for 96 hours in HUVEC-conditioned media. These changes correlated with changes in expression of extracellular matrix degrading enzymes by qRT-PCR. Total RNA was extracted from cells using the RNEasy Mini Plus kit (Qiagen). Complementary DNA was synthesized using 0.5-1 μg RNA and TaqMan reverse transcription reagents (Applied Biosystems). Real-time PCR analysis was performed with an Opticon Real Time PCR Machine (MJ Research) using SYBR Green PCR Master Mix (Applied Biosystems) and appropriate primers. Relative quantifications of gene expression was calculated with standard curves and normalized to GAPDH via the ΔΔCt method. Primer sequences are listed in Table 1.

TABLE 1
RT-PCR Primers.

| Target | forward 5′-3′ | reverse 5′-3′ |
|---|---|---|
| MMP2 | AACGGACAAAGAGTTGGCAG | GTAGTTGGCCACATCTGGGT |
| | (SEQ ID NO: 1) | (SEQ ID NO: 2) |
| MT1-MMP | TGATAAACCCAAAAACCCCA | CCTTCCTCTCGTAGGCAGTG |
| | (SEQ ID NO: 3) | (SEQ ID NO: 4) |
| TIMP1 | GGAATGCACAGTGTTTCCCT | GAAGCCCTTTTCAGAGCCTT |
| | (SEQ ID NO: 5) | (SEQ ID NO: 6) |
| TIMP2 | TGATCCACACACGTTGGTCT | TTTGAGTTGCTTGCAGGATG |
| | (SEQ ID NO: 7) | (SEQ ID NO: 8) |
| Perlecan | ATTCAGGGGAGTACGTGTGC | TAAGCTGCCTCCACGCTTAT |
| | (SEQ ID NO: 9) | (SEQ ID NO: 10) |

As shown in FIG. 9B, expression of pro-invasion genes (MMP2 AND MTI-MMP) in MDA-MB-231 cells decreases and expression of anti-invasive genes (TIMP1, TIMP2) increases in A549 cells after culture for 96 hours in endothelial cell conditioned media, specifically with about a 4-fold decrease in MMP2 gene expression in MD-MB-231 cells and with about a 2-fold increase in gene expression of TIMP1 and TIMP2 in A549 cells (FIG. 9B).

As a control, conditioned media from confluent normal human lung fibroblasts was collected to assess whether the effects observed due to endothelial cell secretions were unique to endothelial cells or whether they were common to other stromal cell types in culture. Media conditioned by healthy fibroblasts had no effect on either cancer cell proliferation (FIG. 9C) or invasiveness (FIG. 9D).

Endothelial cell based suppression of cancer cell invasiveness was accompanied by concomitant changes in expression of matrix modeling genes and known regulators of tumorigenic behavior.

To demonstrate that engrafted endothelial cells also can modulate cancer cell invasiveness in vitro, HUVEC were engrafted on Gelfoam® as described herein and were used to condition media. As shown in FIG. 9E, invasiveness of A549 cancer cells was reduced after 72 hrs of culture in media conditioned with engrafted HUVEC (TEEC).

These data suggest that healthy endothelial cells secrete factors that suppress cancer cell invasiveness.

### Example 4

### Plated Endothelial Cells Modulate Multiple Tumorigenic Pathways

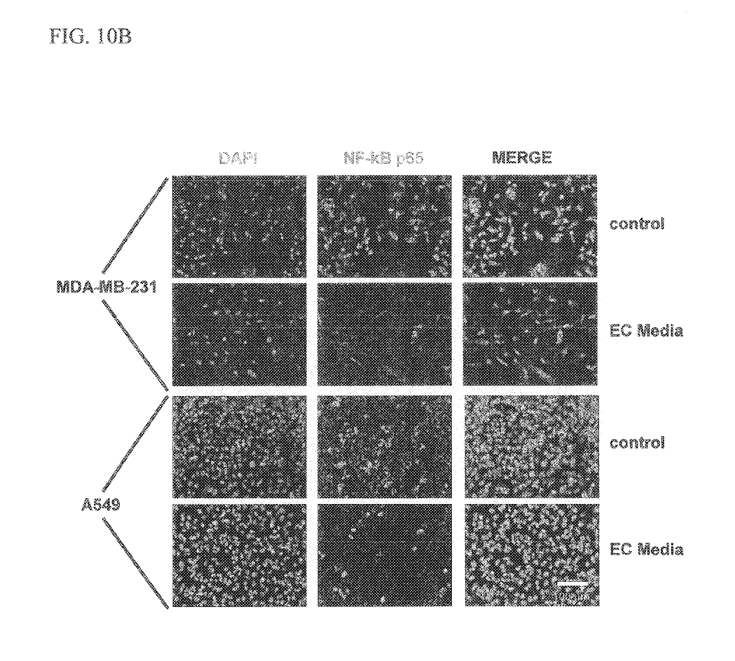

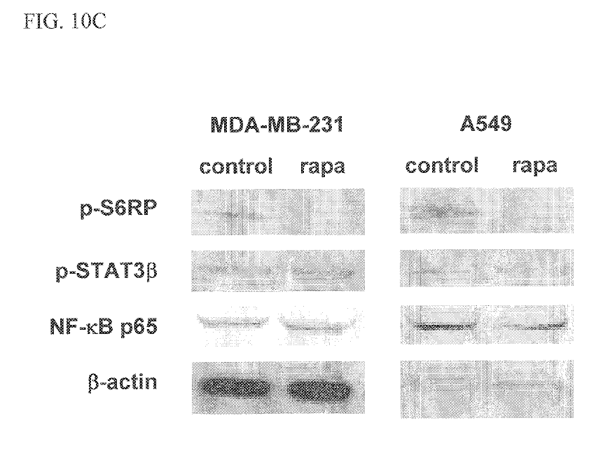

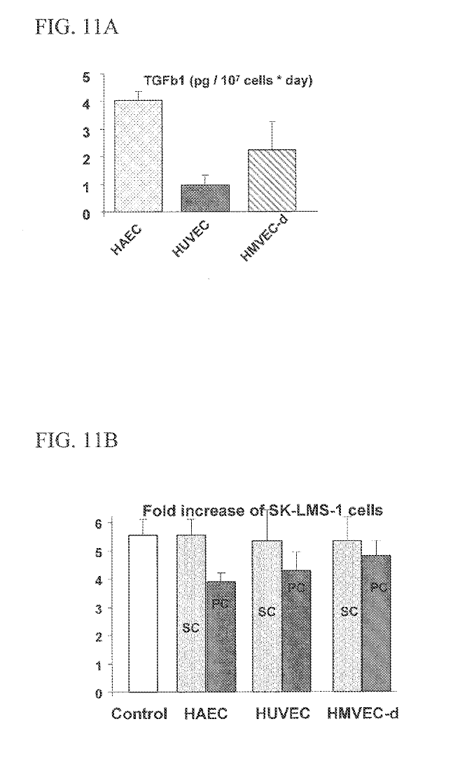

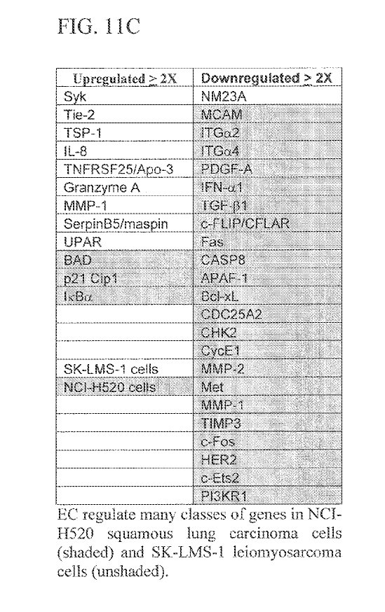

Multiple pro-tumorigenic signaling pathways have been studied and can contribute to both cancer cell proliferation and invasiveness. The S6 ribosomal pathway and two common (and frequently linked) pro-inflammatory pathways that can drive many of the malignant behaviors in cancer cells were assayed. As shown in FIG. 10A, after 4 days (96 hours) of culture in HUVEC-conditioned media, phosphorylation of S6 ribosomal protein (p-S6RB) was decreased approximately 70% phosphorylation of STAT3b was decreased by approximately 20%, and the total levels of NF-κB p65 were decreased by approximately 30% in both MDA-MB-231 and A549 cancer cells after 96 hours of culture in HUVEC-conditioned media, relative to control, as measured by Western blot. Additionally, as shown in FIG. 10B, it was found that the intensity and nuclear localization of NF-κB p65 was decreased by culture in HUVEC-conditioned media in both cell lines using immunofluorescent staining and imaging.

As a control, pharmacological inhibition of S6RP phosphorylation was used to assay any S6RP phosphorylation-specific changes in the expression of STAT3β and NF-κB p65. Pharmacological inhibition was performed using rapamycin, a mTOR inhibitor. As shown in FIG. 10C, complete inhibition of S6RP phosphorylation with rapamycin—at a dose (about 0.13 μg/mL) that slows proliferation to a similar degree as culture in HUVEC-conditioned media after 4 days—did not induce significant changes in the phosphorylation of STAT3β or in the total levels of NF-κB p65 in MDA-MB-231 or A549 cells.

These data suggest that signaling through pro-tumorigenic and inflammatory pathways is attenuated when cancer cells are cultured with secretions from healthy endothelial cells.

### Example 5

### Endothelial Cells Regulate Cancer Cell Phenotype

As shown in FIG. 11A, it was confirmed that HAEC, HUVEC, and HUVEC-d secrete at least TGF-β under the conditions of testing. A standard ELISA kit (Assay Designs, Ann Arbor, Mich.) was used to evaluate whether endothelial cells from different vascular beds differentially secrete TGF-β. Although the presence of this endothelial cell factor does not correlate with the observed effects on cancer cell phenotype described in Example 2, we propose that variable release of other (combination of) endothelial cell-secreted factors will correlate with effects on target cancer cells.

As shown in FIG. 11B, the proliferation (MTS assay) of SK-LMS-1 leiomyosarcoma cells was also inhibited by media conditioned by endothelial cells. In this case factors secreted from postconfluent endothelial cells suppressed cancer cell proliferation. These results confirm that endothelial cells state (subconfluent/activated vs. postconfluent/quiescent) and origin affect ability to regulate behavior of target cancer cells.

In addition, as shown in FIG. 11C, gene expression of NCI-H520 cells and SK-LMS-1 cells cultured for 24 hours in media conditioned by postconfluent aortic endothelial cells was studied using the Cancer Pathway Finder qRT-PCR array (SABiosciences, Baltimore, Md.). Of the 84 genes in the array, ten genes in SK-LMS-1 were up- or down-regulated at least twofold (9 up, 1 down); 25 genes in NCI-H520 were up- or down-regulated (3 up, 22 down) at least twofold. Expression changes in genes important for cell adhesion, angiogenesis, apoptosis and senescence, cell cycle control and DNA damage repair, invasion and metastasis, and other signal transduction molecules were observed. Many genes that positively regulate NCI-H520 survival and proliferation (e.g. Bcl-xL, PI3KRI) were downregulated, whereas genes that negatively regulate survival and proliferation (e.g. BAD, P21Cip1) were upregulated. The gene expression changes in SK-LMS-1 cells were more nuanced. For example, both anti-angiogenic (TSP-1) and pro-angiogenic (IL-8) molecules were upregulated. These findings imply that endothelial cells have pleiotropic paracrine effects on target cancer cells. Hence multiple endothelial cells-secreted factors likely contribute to these phenomena.

To further explore this confirmed role of endothelial cell factors, experiments will be conducted to determine the levels of various endothelial cell derived regulatory factors and to correlate specific factors with changes in cancer cell phenotype. For example, endothelial cell derived factors which regulate vSMC regulation (e.g., HSPG, PGI₂, NO), T cell proliferation (e.g., Il-6, IL-8), and dendritic cell maturation (TGF-β) will be quantified in order to correlate cancer cell phenotype (e.g., proliferation, invasiveness) and gene expression patterns with specific endothelial cell derived factors. Levels of other endothelial factors with regulatory roles in cancer pathogenesis (e.g., CTGF, ET-1) also will be quantified. Furthermore, quantitative RT-PCR, Western blot, and flow cytometry will be used to measure the differences in RNA and protein expression patterns of cancer cells cultured in media conditioned with endothelial cells.

Because many soluble signaling mediators are proteins, total protein secretion will be measured using a BCA assay. Total GAG and HSPG (proteoglycans important as growth factor co-receptors) then will be determined by dimethylene blue reduction before and after treatment with chondroitinase ABC. Prostacyclin, an important vasodilator and regulator of vSMC proliferation, will be measured with a 6-ketoprostaglandin F1 ELISA assay kit. NO, another regulator, will be measured by its stable breakdown products (nitrite and nitrate, Nitric Oxide Assay Kit, Pierce, Rockford. Ill.). TGF-β (which has diverse effects on wound healing and cancer), endothelin (a potent vasoconstrictor and contributor to tumor metastasis), and CTGF will be measured with standard ELISA kits. All biochemical assays and immunoassays will be performed as described above. Subsequently, identified factors will be verified by neutralizing one or more identified factors (e.g., by adding neutralizing antibodies or pharmacologic inhibitors) in the endothelial cell conditioned media prior to addition of cancer cells. Cancer cells will be observed to determine whether cancer cell phenotypes revert in the presence of neutralizing antibodies or pharmacologic inhibitors, thereby indicating that the neutralized factor is a cancer cell modulator.

Control experiments will include quantifying the effects of endothelial cell conditioned media on vSMC proliferation (MTS assay or ³H-thymidine incorporation), T cell proliferation (³-H-thymidine incorporation), and dendritic cell maturation (ELISA for dendritic cell secretion of IL-10, TGF-β; flow cytometry profiling of CD40, CD80, CD86, CD83, HLA-DR expression changes in dendritic cells).

A commercially available qRT-PCR array (RT2 Cancer PathwayFinder, SABiosciences, Baltimore, Md.) will be used to quantify the levels of genes which play important roles in cancer pathogenesis, including genes involved in cell cycle control and DNA damage repair (e.g., p53, mdm2, pRb), apoptosis and cell senescence (e.g., BCL-2, caspase-8, hTERT), adhesion (e.g., integrins αᵥ and β₃, MCAM), angiogenesis (e.g., IL-8, VEGF-A, PDGF-A), and invasion/metastasis (e.g., MMP-2, Twist), as well as other genes with more complex functions (e.g., NF-κB, fos, jun, MEK). Protein expression of identified genes can be verified by Western blot, ELISA, flow cytometry, or any other means well known in the art. These techniques are described in detail above.

### Example 6

### Engrafting of Endothelial Progenitor Cells on a Biocompatible Matrix

Endothelial progenitor cells will be isolated and engrafted within biocompatible matrices to evaluate the ability of endothelial progenitor cells to control cancer cells. Endothelial progenitor cells will be isolated from adult peripheral blood, as described above, and will be cultured in a 3-D gelatin scaffold including but not limited to Gelfoam®, previously shown to support mature endothelial cells and epithelial cells. The expression levels of key regulatory genes will be monitored upon matrix embedding using qRT-PCR (SABiosciences, Baltimore, Md.), Western blot, and flow cytometry to measure the expression of regulators of endothelial “quiescence”. Genes of interest include, but are not limited to, integrins (α6β1, ανβ3, α2β1, α6β1), extracellular matrix (collagen IV, fibronectin), NF-κB (including regulators thereof, e.g., IκB) and downstream targets (e.g., MCP-1, IL-6, IL-8), adhesion molecules (VCAM-1 ICAM-1), and other endothelial regulatory genes (KLF2, KLF4). The paracrine regulatory properties of matrix engrafted endothelial progenitor cells will be compared to the paracrine regulatory properties of matrix engrafted mature endothelial progenitor cells, including the effects of matrix engrafted endothelial progenitor cells on vSMC proliferation, T cell proliferation, and dendritic cell maturation, as describe in Example 1.

It is expected that endothelial progenitor cells cultured on 3-D gelatin scaffolds (i.e., engrafted endothelial progenitor cells) will exhibit similar gene expression changes as have been documented for mature endothelial cells such as those described in Example 3. Thus, it is expected that engrafted endothelial progenitor cells will adopt a quiescent regulatory phenotype characteristic of healthy endothelial cells.

### Example 7

### Immunologic, Pharmacologic and Genetic Manipulation of Endothelial Cells

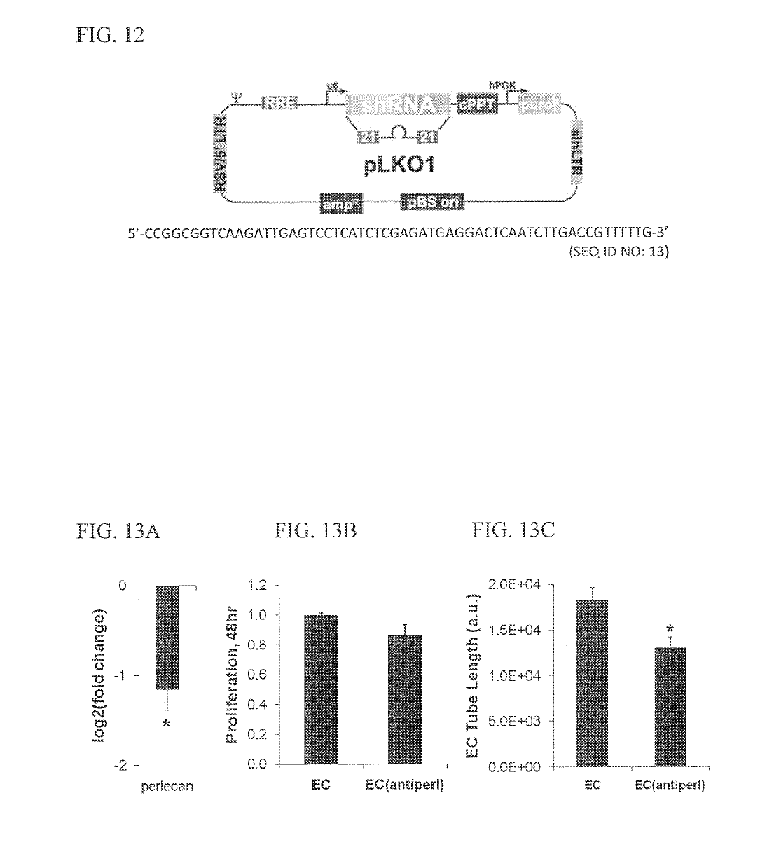

RNA interference was used to modulate the expression of perlecan (a heparan sulfate proteoglycan expressed by HUVEC with diverse cell-signaling effects) by endothelial cells to determine if knockdown of perlecan affects the ability of engrafted endothelial cells to control cancer cell virulence. Lentiviral plasmids containing shRNA against perlecan (and, as a control, the plasmid vector without shRNA) were purchased from Open Biosystems (Huntsville, Ala.). Plasmids were grown in transformed bacteria, isolated (PureLink HiPure Maxiprep system, Invitrogen). Packaging, envelope, and Rev vectors were co-transfected simultaneously as described in Chitalia et al. (2008) Nat Cell Biol 10:1208-1216. Briefly, PPAX2 and GP plasmids coding for the aforementioned vectors were co-transfected, along with the shRNA-bearing plasmid, using Lipofectamine (Invitrogen) into HEK-293T packaging cells. Viral particles were collected for 48 hours and transferred, along with 8 μg/mL hexadimethrine bromide, to subconfluent EC monolayers. Puromycin (1 μg/ml) was used for selection of stably transduced ECs. The commercial lentiviral plasmid construct and shRNA sequence are shown in FIG. 12.

Proliferation was measured by harvesting adherent cells and counting the cell suspension concentration with a Coulter counter (Beckman Coulter, Fullerton, Calif.). In vitro EC tube forming was evaluated by seeding ECs in a 96-well plate (15,000 cells per well) that had been coated with 50 μL of Matrigel (BD Biosciences). After 18-20 hours, tube formation was imaged by phase contrast microscopy. ImageJ software was used to quantify tube length (number of pixels of tubes in the central 20× field of each well), using 4 wells per condition. Perelecan expression levels were assayed by RT-PCT as described in Example 3 using the primers shown in Table 2.

TABLE 2
RT-PCR Primers for Perlecan

| Target | forward 5′-3′ | reverse 5′-3′ |
|---|---|---|
| Perlecan | ATTCAGGGGAGTACGTGTGC | TAAGCTGCCTCCACGCTTAT |
| | (SEQ ID NO: 11) | (SEQ ID NO: 12) |

Perlecan-knockdown EC (ECₐₙₜᵢ₋ₚₑᵣₗ) expressed about 60% less perlecan mRNA than normal EC (qRT-PCR, FIG. 13A). Moderate perlecan knockdown in EC had little to no effect on EC proliferation (FIG. 13B) but modestly reduced their tube-forming capabilities (FIG. 13C), indicating that some normal EC functions may have been altered.

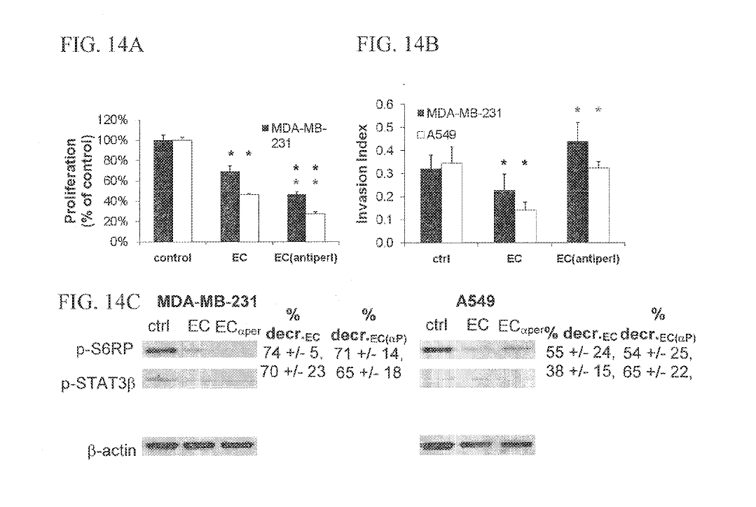

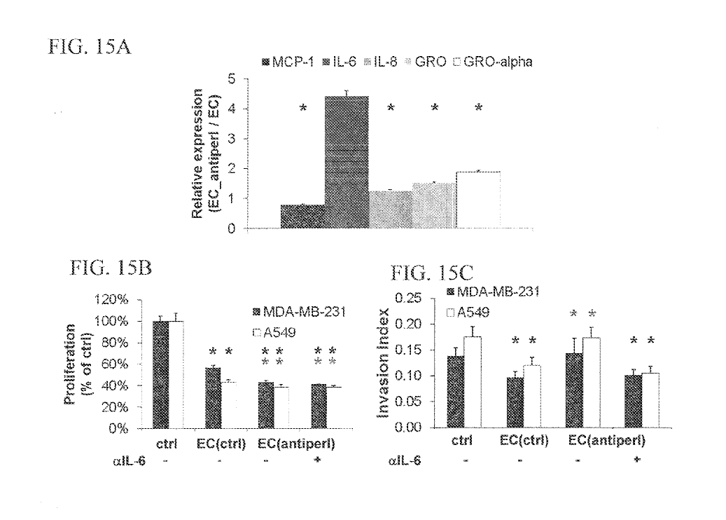

Media conditioned by ECₐₙₜᵢ₋ₚₑᵣₗ had an increased inhibitory effect on cancer cell proliferation compared to media conditioned by HUVEC transduced with the control plasmid (FIG. 14A). However, media conditioned by ECₐₙₜᵢ₋ₚₑᵣₗ could no longer suppress cancer cell invasiveness (FIG. 14B). Secretions from EC with reduced perlecan expression, relative to those from control EC, differently affect expression of pro-tumorigenic/invasive proteins in MDA-MB-231 and A549 cells (FIG. 14C). Significant changes were not seen in the expression of p-S6RP, p- or STAT3β that correlated with these differential effects (FIG. 14C).

Since perlecan can interact directly with many different signaling molecules media conditioned by ECₐₙₜᵢ₋ₚₑᵣₗ was assayed using a cytokine antibody array to determine whether it contained different levels of cytokines. As shown in FIG. 15A, HUVEC with reduced perlecan expression released 4.5 times more interleukin-6 (IL-6) into medium compared with EC transduced with a control plasmid; levels of a few other cytokines were increased but more modestly. Next, it was determined whether the increased IL-6 release from ECₐₙₜᵢ₋ₚₑᵣₗ was directly responsible for the differential effects described earlier. EC-conditioned media was preincubated with 50 μg/mL IL-6 neutralizing antibody or control antibody before transferring it to cancer cell cultures for 4 days and repeating proliferation and invasiveness assays. As shown in FIG. 15B, IL-6 neutralization had no effect on the increased proliferation inhibition of ECₐₙₜᵢ₋ₚₑᵣₗ compared with EC, but completely restored the ability of media conditioned by ECₐₙₜᵢ₋ₚₑᵣₗ to inhibit cancer cell invasiveness (FIG. 15C). These data suggest that increased IL-6 secretion from EC with reduced perlecan expression is responsible for its inability to reduce cancer cell invasiveness.

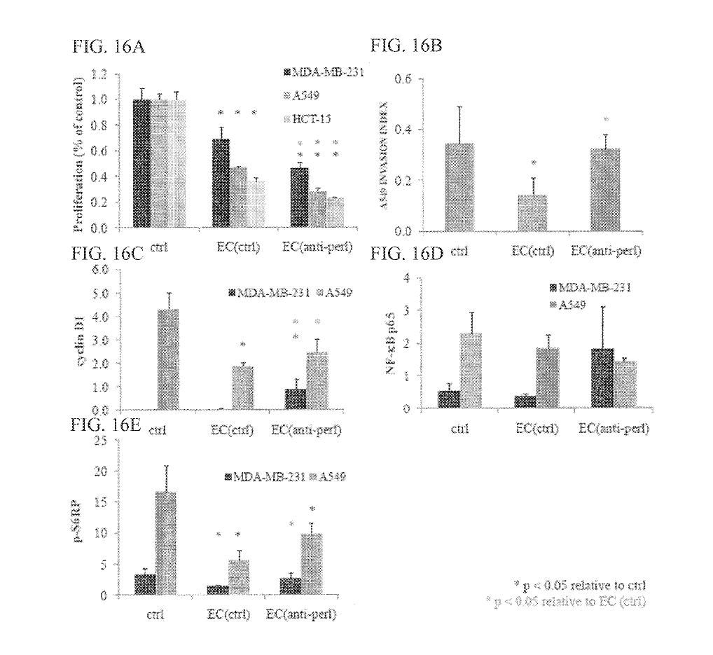

As shown in FIG. 16A, cancer cell proliferation is more strongly inhibited by HUVEC with reduced perlecan expression.

As shown in FIG. 16B, Cancer cell invasiveness is no longer inhibited by HUVEC with reduced perlecan expression.

As shown in FIG. 16C, expression of proliferation proteins in cancer cells is affected differently by HUVEC with reduced perlecan expression.

As shown in FIG. 16D, expression of inflammatory proteins in cancer cells is affected differently by HUVEC with reduced perlecan expression.

As shown in FIG. 16E, expression of p-S6RP in cancer cells is not significantly affected by HUVEC with reduced perlecan expression.

Endothelial cell/substratum units were constructed with genetically modulated levels of key secreted regulatory factors. Endothelial cells transfected with shRNA against perlecan, an endothelial cell HSPG, and an endothelial cell stably transfected with shRNA against heparanase were used to vary the mitogenic signaling associated with HSPG/growth factor shuttling.

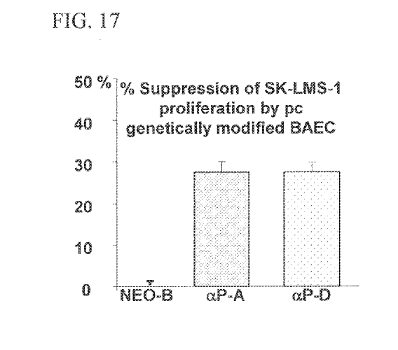

As shown in FIG. 17, SK-LMS-1 proliferation is increased upon exposure to conditioned media from two different postconfluent (PC) clones of BAEC with knocked down perlecan expression (αP-A & D) was more effective than normal BAEC transfected with a nonsense antisense construct (NEO-B).

RNA interference will be used to modulate the expression of other key regulatory factors expressed by endothelial cells to determine if knockdown affects the ability of engrafted endothelial cells to control cancer cell virulence. Briefly, engrafted endothelial cell matrices will be generated with genetically modulated levels of key secreted regulatory factors. For example, HAEC will be stably transfected with shRNA against heparinase to vary the mitogenic signaling associated with HSPG/growth factor shuttling. In addition, knockdown (e.g., Mission shRNA Lentiviral Transduction particle system; Sigma, St. Louis, Mo.) or forced overexpression (e.g., Lentiviral Construction Services; GenScript Corp., Piscataway, N.J.) will be used to modulate the levels of other endothelial cell factors (e.g., connective tissue growth factor (CTGF), transforming growth factor β1 (TGF-β1) identified in Examples 2-6, to verify that these factors play direct regulatory roles in controlling cancer cell virulence. Immunoglobulins (e.g., antibodies) and pharmacologic compounds also will be used to inhibit specific endothelial cell derived factors at the protein level.

It is expected that genetically or pharmacologically varying the secretion of certain (classes of) endothelial secreted factors will affect the ability of engrafted endothelial cells to regulate target (cancer) cell virulence (proliferation, invasiveness, and gene expression controlling these and other properties).

### Example 8

### Cancer Cell Types/States Show Differential Susceptibility to Endothelial Cell Control

Various cancer cell lines will be used to evaluate how cancer differentiation state and tissue origin affect the susceptibility of cancer cells to control by cell engrafted biocompatible matrices of the present teachings. Using methods as described above, various cancer cell lines, such as those listed in Table 3, will be examined for their response to media conditioned by cell engrafted biocompatible matrices of the present teachings. Specifically, media conditioned with engrafted cells is examined for its affect on cancer cell proliferation (cell cycle progression and survival) and invasiveness, as well as to correlate changes in cancer gene expression with phenotypic changes. Differential gene expression is verified at the protein level by Western blot, ELISA, flow cytometry, and other methods well known in the art. Because cancer cell types can respond differently to endothelial cell control, additional cancer cell types (e.g., lineage, class, and/or differentiation state) will be tested. In addition, fresh cancer cells will be isolated from primary tumor samples to reduce the impact of any cell line artifacts.

TABLE 3
Cancer cells lines of varying differentiation and tissue origin.

| | ORGAN/ | WELL- | POORLY- |
|---|---|---|---|
| ORIGIN | TISSUE | DIFF’D | DIFF'D |
| EPITHELIAL | lung | NCI-H520 | A549 |
| | breast | MCF7 | MDA-MB-231 |
| | colon | HCT-15 | HCT-116 |
| MESENCHYMAL | smooth muscle | SK-LMS-1 | SK-UT-1 |
| | bone | U-2 OS | SK-ES-1 |
| HEMATOPOIETIC | myeloid cells | KU812 | Kasumi-1 |
| NEUROEPITHELIAL | astrocytes | SW-1088 | U-87 |
| | cerebellum | D283 Med | Daoy |

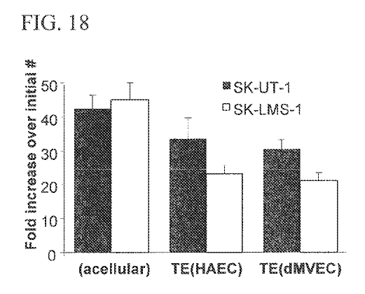

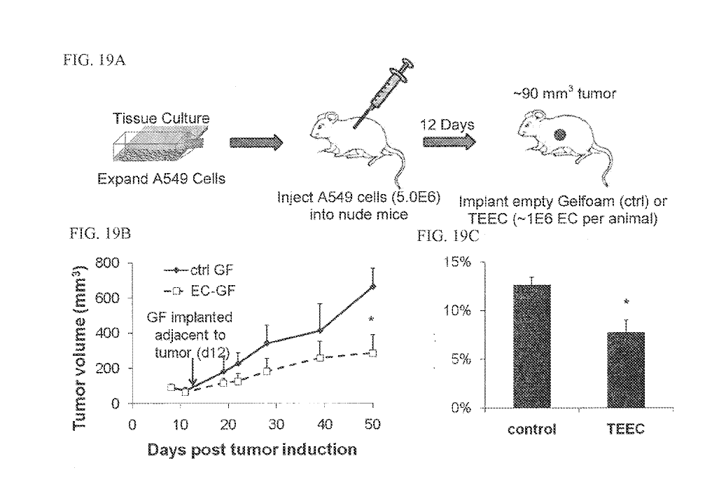

In the case of SK-UT-1 cells, experiments are completed and the data are set forth in FIG. 18. As shown in FIG. 18, six days of culture in media conditioned with engrafted endothelial cell caused a larger decrease in the proliferation of well-differentiated SK-LMS-1 leiomyosarcoma cells, via MTS assay, than poorly-differentiated SK-UT-1 leiomyosarcoma cells.

It is therefore expected that cancer cells of differing tissue origin and differentiation state will show differential susceptibility to endothelial cell control, with epithelial cancers (e.g., carcinoma) showing the most susceptibility to endothelial cell paracrine control of growth and invasiveness.

It is also expected that well-differentiated cancer cells, which more closely resemble the tissue of origin, will be more susceptible to endothelial cell control.

### Example 9

### Ability of Endothelial Cells to Inhibit Cancer Cell Proliferation Under Conditions of Hypoxia (Low Oxygen Tension)

Cancer cells will be cultured in low-oxygen incubators and will be analyzed as described above to assess whether hypoxia affects cancer cell response to endothelial cell conditioned media. For those cancer cell lines with the poorest and those with the most pronounced responses to endothelial cell control, oxygen tension will be varied to assess whether hypoxia mitigates or enhances the ability of cell engrafted biocompatible matrices to control cancer cells. Hypoxia (about 2% O₂ (see, e.g., Denko, “Hypoxia, HIF) and glucose metabolism in the solid tumour,” Nat. Rev. Cancer, 8:705-713 (2008) will be induced either by culture in oxygen-impermeable vacuum Mylar® bags in standard incubators (Petaka/Celartia, Powell, Ohio) or by culture in cell culture chambers with controllable oxygen partial pressure (proOxC™ chamber, BioSherix, Lacona, N.Y.). We will use gene expression studies (qRT-PCR) as discussed above to assess differences in hypoxic cancer cell regulation by endothelial cells. This will yield insight on the convergence of signaling pathways (ligand/receptor binding and hypoxia pathways) that independently affect tumor behavior. A number of oxygen tensions will be used to empirically determine a range of oxygen tensions that modulates endothelial cells control of cancer cells.

It is expected that, since intratumoral hypoxia correlates with poor patient prognosis and can directly induce expression of virulence genes in cancer cells (including cancer stem cells), cancer cells exposed to hypoxic conditions will be less susceptible to endothelial control. The knowledge gained by these experiments may allow us to genetically or pharmacologically modulate engrafted endothelial cell secretion in order to better control cancer cell virulence under conditions of hypoxia (which normally increases tumor virulence).

### Example 10

### Identification of Endothelial Cell Derived Factors Which Inhibit Cancer Cells

Endothelial cell derived factors will be analyzed to determine whether there is a correlation between cancer cell lines and culture conditions that demonstrate the strongest and weakest susceptibility to endothelial cell control. In addition, using the methods described above, it will be determined if specific endothelial secreted factors exert differential effects on cancer lines of differing origins. For example, neutralizing antibodies (e.g., chicken anti-human polyclonal antibody to human TGF-β, Abcam, Cambridge, Mass.) will be added to conditioned media prior to culturing cancer cells, and/or conditioned media is treated with pharmacologic inhibitors of specific receptors (e.g., TGF-β receptor 1 inhibitor, EMD Biosciences, Gibbstown, N.J.). Thereafter, specific gene or protein expression (or activation) changes in cancer cell phenotypes (e.g., SMAD 2/3 phosphorylation) will be assayed as markers of inhibition. Proliferation and invasiveness assays, as described above, will be used as functional correlates. Finally, genetically-modified endothelial cells will be used to verify the direct roles of specific endothelial secreted products in controlling the virulence of a wide range of cancer states (of variable origin, differentiation state, and oxygenation status).

It is expected that highly virulent cancer cells (including poorly-differentiated cells and cells cultured under hypoxia conditions) will show the strongest resistance to endothelial cell control by “ignoring” factors secreted by endothelial cells. However, endothelial factors that control highly-virulent cancer cells in culture will likely control a wider range of cancer states.

### Example 11

### Identification of Genes Differentially Expressed by Engrafted Endothelial Cells in Response to Cancer Cells

Microvascular endothelial cells will be isolated from xenograft tumors to determine whether media conditioned with these microvascular endothelial cells controls cancer cell proliferation and invasiveness in vitro. Briefly, microvascular endothelial cells from murine xenograft tumors will be grown in immunocompromised mice. Tumors are initiated by first expanding cancer cells in culture and subsequently injecting 10⁷ viable cells, suspended in 0.1 mL saline, subcutaneously into the lateral thoraces of mice. After the tumors grow to an average size of about 2000 mm³, animals will be sacrificed and tissues will be collected for cell harvesting as described in van Beijnum, et al, “Isolation of endothelial cells from fresh tissues,” Nat. Protoc., 3(6):085-91 (2008). Briefly, the protocol involves tumor tissue mechanical homogenization, specific antibody labeling of endothelial cells, and magnetic bead separation of labeled cells. Endothelial identity of isolated cells will be confirmed with in vitro functional analyses (Angiogenesis Tube Formation Assay Kit; Millipore, Billerica, Mass.) and analyses of endothelial markers (e.g., flow cytometry for vWF, PECAM, CTGF, SPARC/osteomectin as described by St Croix, et al., “Genes expressed in human tumor endothelium,” Science, 289(5482): 1197-202 (2000). mRNA is isolated from these cells and will be analyzed using a medium-throughput qRT-PCR array (Endothelial Cell Biology PCR Array; SABiosciences, Baltimore, Md.) to quantify gene expression differences between (1) tumor derived microvascular endothelial cells, (2) normal dermal microvascular endothelial cells isolated from the same (tumor-bearing) animals, and (3) dermal microvascular endothelial cells isolated from animals without tumors.

In addition, tumor derived microvascular endothelial cells will be engrafted on biocompatible matrices and will be cultured in vitro, in accordance with present teachings. The media conditioned with engrafted tumor derived microvascular endothelial cells subsequently will be used to grow cancer cells, to determine if the conditioned media affects cancer cell proliferation, and gene/protein expression. Endothelial genes that are identified as significantly upregulated or downregulated by cancer cell conditioned media also will be correlated to functional differences in the ability of pretreated endothelial cells to control cancer virulence. Immunoglobulin, pharmacologic or genetic manipulations will be used to confirm endothelial cell-expressed genes that are tumor or virulence promoters.

It is expected that endothelial cells isolated from tumor microvasculature are programmed in such a way that they promote tumor virulence rather than inhibit tumor virulence. The identification of cancer cell derived factors or endothelial cell derived factors responsible for tumor promotion will permit neutralization of these factors, as described above, thereby preventing the engrafted endothelial cells from become tumor promoters.

### Example 12

### Endothelial Cell Engrafted Biocompatible Matrices Suppress Cancer Proliferation In Vitro

24 Crl:NU-Foxnl female mice, 6 to 8 weeks of age, were injected with cancer cells.

The human lung carcinoma cell line, A549, was obtained from American Type Culture Collection (ATCC; catalog number: CCL-185™). A549 cells were cultured in Dulbecco's Modified Eagles Medium (DMEM), supplemented with 4 mM L-glutamine, 0.1 mM non-essential amino acids, 10% fetal bovine serum (FBS), or other appropriate medium. Cells were incubated in 5 CO₂ and =70% humidified air at 35° C. to 39° C.

Exponentially growing cells were harvested, washed twice in Hank's Balanced Salt Solution (HBSS) to remove any traces of trypsin or serum. Percent of cells viable and total viable cells were determined before injection by using the trypan blue exclusion method. Cells were suspended in Hanks Balanced Salt Solution (HBSS) for injections.

Each of the 24 study animals received 1.0×10⁷ viable A549 cells injected subcutaneously in the right lateral thorax. The cells were injected at a concentration of 1.0×10⁸ viable cells per mL. Each animal received 0.1 mL of this cell suspension.

Human Aortic Endothelial Cells were embedded in a gelatin matrix, Gelfoam®, HAEC engrafted Gelfoam® were stored in an insulated container at ambient temperature (15-30° C.) and protected from light, with approximately 75 mg of particles in a 50 mL conical tube with 35 mL media. The final concentration (in a syringe) of HAEC engrafted Gelfoam® was approximately 25 mg/mL. Approximately 500 μL (approximately 12.5 mg) was injected in each animal using a 21 gauge needle (or larger).

HAEC engrafted Gelfoam® particles were transferred from a 50 mL conical tube to a syringe for injection. Media was expelled, leaving approximately 2 mL media remaining with the particles in the syringe. 1-2 mL of saline was added to the syringe and additional media was expelled to obtain a final concentration of 25 mg/mL.

To prepare controls, empty (non-cell engrafted) Gelfoam® particles were stored in an insulated container at ambient temperature (15-30° C.) and protected from light, with approximately 75 mg of particles in a 50 mL conical tube with 35 mL of transport media. The final concentration (in a syringe) of empty Gelfoam® particles was approximately 25 mg/mL. Approximately 500 μL (approximately 12.5 mg) was injected in each animal using a 21 gauge needle (or larger).

Empty Gelfoam® particles and a minimal amount of transport media were transferred from a 50 mL conical tube to a syringe for injection. Transport media was expelled, leaving approximately 2 mL of transport media remaining with the particles in the syringe. 1 mL of saline was added for a final volume of 3 mL. If possible, additional transport media was expelled to obtain a final concentration of 25 mg/mL.

As shown in Table 4, each of the three study groups contained 4 mice. Group 1 was untreated. Group 2 contained the Vehicle Control treated mice. Group 3 contained Test Article treated animals. When the calculated mean weight of 12 tumors, 1 tumor in each of 12 different animals, reached a target window size of approximately 100-200 mg, the animals were sorted into one of the three study groups using block randomization based on the calculated tumor weight. Animals then received the indicated treatment. When the calculated mean weight of 12 tumors reaches a target window size of approximately 300-400 mg the animals were sorted into one of the three study groups using block randomization based on the calculated tumor weights. Animals then received the indicated treatment.

TABLE 4
Treatment Groups

| | | | | | Dose | | |
|---|---|---|---|---|---|---|---|
| | | | Dose | Concentration | Volume | | Dose |
| Groups | N | Compound | mg/kg | mg/mL | mL/kg | Dose Route | Schedule |
| 1 | 4 | Untreated | N/A | N/A | N/A | N/A | N/A |
| 2 | 4 | Gelfoam ® | 625 mg/kg | 25 mg/mL | 25 mL/kg | SC | once |
| | | Control | | | | (intrascapular; | |
| | | | | | | near tumor | |
| | | | | | | placement) | |
| 3 | 4 | HAEC/ | 625 mg/kg | 25 mg/mL | 25 mL/kg | SC | once |
| | | Gelfoam ® | | | | (intrascapular; | |
| | | | | | | near tumor | |
| | | | | | | placement) | |

The injection site of each animal was monitored twice weekly for signs of tumor growth. Throughout the study, the length (L) and width (W) of any tumors that developed were measured in millimeters using calibrated vernier calipers, where L is the longer of the two (2) dimensions.

When applicable, tumor weight (M) in milligrams was calculated by using the formula associated with a prolate ellipsoid: M=(L×W²)/2. Individual animal weights were taken twice a week throughout the course of this study.

An interim blood sample was collected via submandibular facial vein before tumor implantation (baseline), following group sorting just prior to initial dose administration, and at the end of the study. Blood was collected into K2 EDTA tubes. A maximum of 100 μL of whole blood was collected from each study animal. Whole blood samples were stored at 5±3° C. on cold packs during delivery.

At the end of the study, animals were euthanized via carbon dioxide inhalation and blood was collected into K2 EDTA tubes, stored at 5±3° C. during transport. The following tissues were collected, weighted and placed into 4% paraformaldehyde: Tumor and implant site with surrounding tissue. Tissues were paraffin-embedded and sectioned (5 μm) without staining.

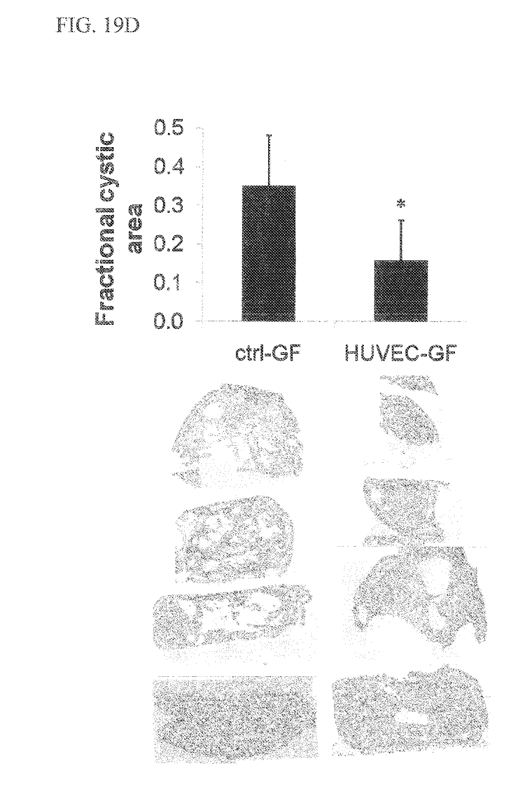

As shown in FIG. 19B, cell engrafted biocompatible matrices suppressed cancer proliferation in vivo. 10⁷ exponentially-growing A549 (large cell lung carcinoma) cells were injected subcutaneously into the thoraces of 6-8 week old female nude mice. After allowing about 7 days for engrafted tumors to reach an average size of 100 mm³, either empty Gelfoam® particles or HAEC-Gelfoam® particles (625 mg/kg) were injected subcutaneously adjacent to the tumor. Tumor mass (assuming a density of 1 mg/mm³) was estimated by two caliper measurements during the indicated days. Gelfoam® particles (and embedded cells) were resorbed in about 10 days.

As shown in FIG. 19C, tumor growth inhibition was correlated with a decrease in the fraction of Ki-67 positive cancer cells within the tumor after cryosectioning and immunofluorescent staining. As shown in FIG. 19D, tumor growth inhibition was also correlated with a decrease in the fraction of the tumor filled with cysts.

Additionally, cell engrafted planar biocompatible matrices will be implanted adjacent to primary tumors in vivo to examine their effects on tumor growth, local invasion, and distant metastasis in murine cancer models (see FIG. 19A). Briefly, optimal cell engrafted planar biocompatible matrices will be cultured for 1-2 weeks in vitro (as described in the Reference Example 1) and subsequently implanted, either adjacent to the primary tumor (paracrine regulation) or intraperitoneally (endocrine). Controls include implantations of empty (cell-free) hydrated Gelfoam® planar biocompatible matrices and administration of sham surgery with no implants (i.e., untreated). Tumor volume will be estimated serially by caliper measurements. After 3-4 weeks, or when tumors reach ˜2000 mm³ in volume, animals will be euthanized and primary tumors excised and weighed. Blood will be collected at sacrifice by cardiac puncture and analyzed for circulating cancer cells and endothelial progenitor cells by flow cytometry. Blood collected post-sacrifice will be compared to blood drawn, either from the tail vein or retroorbital plexus, before the cancer implantation (day 0) as a control. Primary tumors and adjacent tissues will be paraffin-embedded, sectioned and analyzed for primary tumor histology (H&E), proliferation (Ki67/PCNA), apoptosis (TUNEL), local invasion (EpCAM for lung carcinoma, CD133 for lung cancer stem cells, CD44 for leiomyosarcoma), stroma (macrophages via CALTAG laboratories anti-F4/80 rat monoclonal antibodies, myofibroblasts via α-SMA monoclonal antibodies) and local vascular networks (CD31/PECAM or vWF). Changes will also be analyzed in specific genes identified in the in vitro experiments described above.

A murine tumor metastasis model will also be used to analyze the effect of cell engrafted biocompatible matrices on metastatic cell behavior. Lewis Lung carcinoma (LLC) cells will be injected subcutaneously into the backs of syngeneic immunocompetent mice. After a primary tumor grows to ˜100 mm³, the tumor will be resected in order to allow lung metastases (seeded during primary tumor growth) to develop, as described by O'Reilly, et al., “Angiostatin: a novel angiogenesis inhibitor that mediates the suppression of metastases by a Lewis lung carcinoma,” Cell, 79(2):315-28 (1994). The effects of cell engrafted biocompatible matrices, as implants adjacent to primary LLC tumors, on both primary tumor behavior and metastasis behavior (number and size of lung metastases, metastases to other sites (e.g., bone marrow)) will be studied using histopathological techniques. Circulating levels of cancer cells and endothelial progenitor cells will be determined. The second metastasis model will involve tail vein injection of cancer cells. After determining sites of colonization following hematogenous dissemination, cell engrafted biocompatible matrices will be implanted adjacent to predicted colonized sites to study the effects of metastatic colonization.

The use of headings and sections in the application is not meant to limit the invention; each section can apply to any aspect, embodiment, or feature of the invention.

Throughout the application, where compositions are described as having, including, or comprising specific components, or where processes are described as having, including or comprising specific process steps, it is contemplated that compositions of the invention also consist essentially of, or consist of, the recited components, and that the processes of the invention also consist essentially of, or consist of, the recited process steps.

In the application, where an element or component is said to be included in and/or selected from a list of recited elements or components, it should be understood that the element or component can be any one of the recited elements or components, or can be selected from a group consisting of two or more of the recited elements or components. Further, it should be understood that elements and/or features of a composition, an apparatus, or a method described herein can be combined in a variety of ways without departing from the spirit and scope of the invention, whether explicit or implicit herein.

The use of the terms “include,” “including,” “have,” “has,” or “having” should be generally understood as open-ended and non-limiting unless specifically stated otherwise.

The use of the singular herein includes the plural (and vice versa) unless specifically stated otherwise. Moreover, the singular forms “a,” “an,” and “the” include plural forms unless the context clearly dictates otherwise. In addition, where the use of the term “about” is before a quantitative value, the present teachings also include the specific quantitative value itself, unless specifically stated otherwise.

It should be understood that the order of steps or order for performing certain actions is immaterial so long as the invention remains operable. Moreover, two or more steps or actions may be conducted simultaneously.

Where a range or list of values is provided, each intervening value between the upper and lower limits of that range or list of values is individually contemplated and is encompassed within the invention as if each value were specifically enumerated herein. In addition, smaller ranges between and including the upper and lower limits of a given range are contemplated and encompassed within the invention. The listing of exemplary values or ranges is not a disclaimer of other values or ranges between and including the upper and lower limits of a given range.

The aspect, embodiments, features, and examples of the invention are to be considered illustrative in all respects and are not intended to limit the invention, the scope of which is defined only by the claims. Other embodiments, modifications, and usages will be apparent to those skilled in the art without departing from the spirit and scope of the claimed invention, and all such variations that come within the meaning and range of equivalents are intended to be embraced by the claims.

What is claimed is:

1-44. (canceled)

45. A method of inhibiting a solid tumor in a subject, the method comprising the steps of:
providing an implantable material in an amount effective to inhibit a tumor, the implantable material comprising endothelial cells engrafted on or within a biocompatible matrix; and
implanting an effective amount of the implantable material adjacent a tumor in the subject,
wherein tumor virulence is inhibited as compared to a tumor in an untreated subject.

46. The method of claim 45, wherein inhibiting virulence of a tumor comprises inhibiting tumor cell proliferation.

47. The method of claim 45, wherein inhibiting virulence of a tumor comprises inhibiting tumor cell metastasis.

48. The method of claim 45, wherein inhibiting virulence of a tumor comprises inhibiting invasiveness and colonization.

49. The method of claim 45, wherein inhibiting virulence of a tumor comprises inhibiting secondary tumor growth.

50. The method of claim 45, wherein the tumor is lung cancer.

51. The method of claim 45, wherein the tumor is breast cancer.

52. The method of claim 45, wherein the tumor is colon cancer.

53. The method of claim 45, wherein the implanting step comprises implanting the implantable material such that it contacts a least a portion of an interior or an exterior surface of the tumor.

54. The method of claim 45, wherein the endothelial cells are human aortic endothelial cells.

55. The method of claim 45, wherein the endothelial cells are human umbilical vein endothelial cells.

56. The method of claim 45, wherein the implantable material comprises xenogeneic endothelial cells engrafted on or within the matrix.

57. The method of claim 45, wherein the endothelial cells comprise endothelial progenitor cells.

58. The method of claim 45, wherein the implantable material inhibits virulence of the tumor by paracrine regulation.


SEQUENCE LISTING (13 sequences)

SEQ ID NO: 1 - DNA, 20 bp, Artificial Sequence
  primer
aacggacaaagagttggcag

SEQ ID NO: 2 - DNA, 20 bp, Artificial Sequence
  primer
gtagttggccacatctgggt

SEQ ID NO: 3 - DNA, 20 bp, Artificial Sequence
  primer
tgataaacccaaaaacccca

SEQ ID NO: 4 - DNA, 20 bp, Artificial Sequence
  primer
ccttcctctcgtaggcagtg

SEQ ID NO: 5 - DNA, 20 bp, Artificial Sequence
  primer
ggaatgcacagtgtttccct

SEQ ID NO: 6 - DNA, 20 bp, Artificial Sequence
  primer
gaagcccttttcagagcctt

SEQ ID NO: 7 - DNA, 20 bp, Artificial Sequence
  primer
tgatccacacacgttggtct

SEQ ID NO: 8 - DNA, 20 bp, Artificial Sequence
  primer
tttgagttgcttgcaggatg

SEQ ID NO: 9 - DNA, 20 bp, Artificial Sequence
  primer
attcaggggagtacgtgtgc

SEQ ID NO: 10 - DNA, 20 bp, Artificial Sequence
  primer
taagctgcctccacgcttat

SEQ ID NO: 11 - DNA, 20 bp, Artificial Sequence
  primer
attcaggggagtacgtgtgc

SEQ ID NO: 12 - DNA, 20 bp, Artificial Sequence
  primer
taagctgcctccacgcttat

SEQ ID NO: 13 - DNA, 58 bp, Artificial Sequence
  shRNA
ccggcggtcaagattgagtcctcatctcgagatgaggactcaatcttgaccgtttttg